(19) United States
(12) Patent Application Publication
(10) Pub. No.: US 2025/0282810 A1
(43) Pub. Date: Sep. 11, 2025

(54) Chemical Synthesis Method and Application of V. Cholerae Serotype O100 O-Antigen Oligosaccharides

(71) Applicant: Jiangnan University, Wuxi (CN)

(72) Inventors: Jian YIN, Wuxi (CN); Jing HU, Wuxi (CN); Guodong CHEN, Wuxi (CN)

(21) Appl. No.: 19/216,877

(22) Filed: May 23, 2025

Related U.S. Application Data
(63) Continuation of application No. PCT/CN2024/127376, filed on Oct. 25, 2024.

(30) Foreign Application Priority Data
Jun. 19, 2024 (CN) 2024107916629

(51) Int. Cl. C07H 15/12; C07H 1/00

(52) U.S. Cl. CPC C07H 15/12; C07H 1/00

(57) ABSTRACT

The disclosure discloses a chemical synthesis method and an application of V. cholerae serotype O100 O-antigen oligosaccharides, belonging to the field of chemical technologies. The disclosure uses three monosaccharide building blocks and five carboxylic acid derivatives to synthesize five oligosaccharide fragments of V. cholerae serotype O100 O-antigen under the action of the solvent effect, temperature effect, neighboring group participation effect, etc., through orthogonal protection, selective assembly and amide coupling. The absolute configurations and immunological effects of 3,5-dihydroxyhexanoyl in the O-antigen trisaccharide are illustrated by the synthesized oligosaccharide fragments in combination with the NMR analysis and glycan microarray technology, thereby providing a theoretical basis for further structure-activity study and minimal antigenic epitope screening. The disclosure has excellent application prospects in the aspects of development of V. cholerae synthetic glycoconjugate vaccines and new drugs, etc.

### TECHNICAL FIELD

The disclosure relates to a chemical synthesis method and an application of V. cholerae serotype O100 O-antigen oligosaccharides, belonging to the field of chemical technologies.

### BACKGROUND

V. cholerae, the pathogen of cholera, may cause a cholera epidemic. Cholera is an acute diarrheal disease characterized by watery diarrhea and potentially fatal dehydration (Qadri et al. Clin. Microbiol. Rev. 2022, 35 (3), e00211-00221). According to the WHO's epidemic briefing, 30 countries reported cholera cases in 2023, and reported 40,900 cases and 775 death cases in January, 2024 alone.

Antibiotics and oral cholera vaccines (OCVs) have been widely used to treat V. cholerae infection. Research has shown that V. cholerae has developed widespread resistance to all of these antibiotic drugs. Although OCVs have been proven effective, they also have many limitations, including poor immunogenicity in young children, short protection period, and delayed immune induction (Waldor et al. Annu. Rev. Microbiol. 2022, 76 (1), 681-702). At present, there is still a need for a cholera vaccine that can play a key role in global cholera control.

Glycoprotein conjugate vaccines have shown promising prospects. There are already multiple glycoprotein conjugate vaccines on the market for disease prevention and treatment, such as a 13-valent pneumococcal glycoconjugate vaccine, a meningitic glycoconjugate vaccine, and a Salmonella conjugate vaccine. The use of structurally defined synthetic oligosaccharides coupled with protein carriers can enhance the immunogenicity of oligosaccharides and induce T cells to generate a dependent immune reaction, which has been proven to be safe and reliable (Seeberger, Chem. Rev. 2021, 121 (7), 3598-3626).

According to different surface lipopolysaccharide O-antigens of bacteria, V. cholerae is classified into more than 200 serotypes. In 2019, Perepelov. et al. isolated and characterized the structure of the trisaccharide repeating unit of V. cholerae serotype O100 O-antigen as [→3)-β-d-QuipNAc4N(dHh)-(1→3)-α-d-Fucp4N(RHb)-(1→3)-α-l-FucpNAc-(1→], where RHb and dHh represent (R)-3-hydroxybutanoyl and 3,5-dihydroxyhexanoyl groups, respectively (Perepelov. et al. Carbohydr. Res. 2019, 472, 98-102).

The O-antigen trisaccharide contains two 1,2-cis-α-glycosidic linkages difficult to construct and one 1-2-trans-β-D-quinovosidic linkage easy to break, and four nitrogen atoms are coupled with two rare modifying groups. Due to the presence of the above factors, the synthesis of the O-antigen trisaccharide repeating unit is extremely challenging, and it has not yet been fully synthesized. It is worth noting that bacterial surface polysaccharide modifying groups are considered as potential immune targets. The absolute configurations of two centers of chirality in a unique dHh modifying group remain unclear, and there is a lack of understanding of immunological effects thereof. The chemical synthesis of structurally homogeneous O-antigen oligosaccharides, and the completion of assignment of the absolute configuration and the preliminary immunological study are of great significance for the development of V. cholerae serotype O100 glycoconjugate vaccines and related drugs.

### SUMMARY

### Technical Problem

In view of the above problems, the disclosure relates to development of a chemical synthesis method of V. cholerae serotype O100 O-antigen oligosaccharides, and an application of synthesized oligosaccharides to illustrate the absolute configurations and immunological effects of dHh modification groups.

### Technical Solution

Due to the uncertainty of the absolute configurations of two centers of chirality in dHh, theoretically, there are four potential isomers of the O-antigen trisaccharide. Furthermore, in order to facilitate the study of the immunological effects of dHh, it is necessary to synthesize a trisaccharide derivative without dHh modifications. A corresponding linker is introduced at the reducing end of the synthetic oligosaccharide, so as to provide a basis for further immunological exploration. Therefore, the disclosure uses three monosaccharide building blocks and five carboxylic acid derivatives to synthesize four potential trisaccharide isomers and one derivative through a series of orthogonal protection, selective assembly, and amide coupling. An NMR technology is used to perform error analysis between four trisaccharide isomers and extracted lipopolysaccharide O-antigens to illustrate the absolute configuration of dHh. Five oligosaccharide fragments are bound to a microarray to prepare a glycan microarray, and the immunological effects of dHh are screened and evaluated through the glycan microarray.

One objective of the disclosure is to provide a chemical synthesis method of V. cholerae serotype O100 O-antigen oligosaccharides. The method uses three monosaccharide building blocks and five carboxylic acid derivatives as raw materials;

the structures of the V. cholerae serotype O100 O-antigen oligosaccharides are represented by the following Formulae (1) to (5):

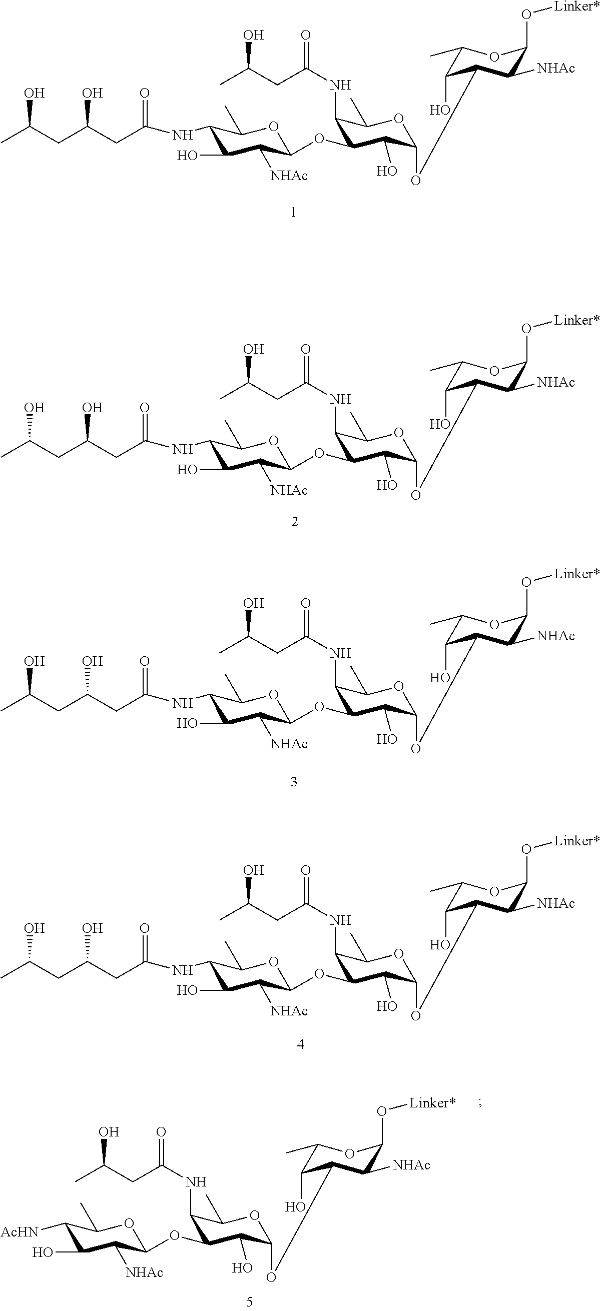

the structures of the three monosaccharide building blocks are respectively represented by Formulae (6) to (8), and the structures of the five carboxylic acid derivatives are respectively represented by Formulae (9) to (13):

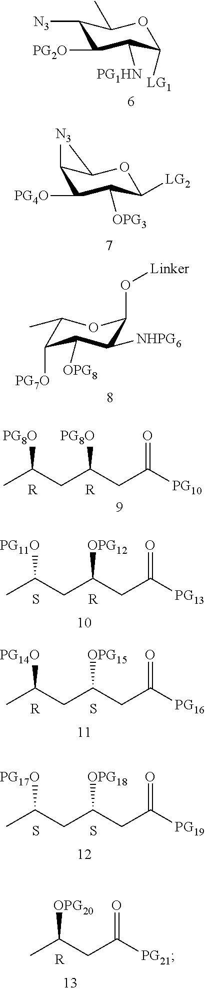

PG₂, PG₃, PG₄, PG₆, and PG₇ are temporary hydroxyl protecting groups which may be one of benzyl (Bn), 2-naphthylidene (Nap), tert-butyldimethylsilyl (TBS), tert-butyldiphenylsilyl (TBDPS), and triethylsilyl (TES);
PG₈, PG₉, PG₁₁, PG₁₂, PG₁₄, PG₁₅, PG₁₇, PG₁₈, and PG₂₀ are temporary hydroxyl protecting groups which may be one of benzyl (Bn), 2-naphthylidene (Nap), acetyl (Ac), benzoyl (Bz), neopentyl, 9-pentamethoxycarbonyl (Fmoc), and 2-p-methoxybenzyl (PMB);
PG₁₀, PG₁₃, PG₁₆, PG₁₉, and PG₂₁ are each independently selected from one of hydroxyl (OH), chlorine (Cl), bromine (Br), fluorine (F), and C1-4 alkoxy;
PG₁ is a temporary amino protecting group which may be one of trichloroacetyl (TCA), dichloroacetyl (DCA), and chloracetyl (ClAc);
PG₅ is a temporary amino protecting group which may be one of acetyl (Ac), trichloroacetyl (TCA), dichloroacetyl (DCA), chloracetyl (ClAc), trichloroethoxycarbonyl (Troc), phthaloyl (Phth), 9-fluorenylmethoxycarbonyl (Fmoc), and tert-butyloxycarbonyl (Boc);
Linker is —(CH₂)ₙN—Y₁Y₂ or —(CH)S—Y₁, where n=1-25, and Y₁ and Y₂ are one of hydrogen, acyl, benzyl (Bn), 2-naphthylmethyl (Nap), and benzylmethoxycarbonyl (Cbz); Linker* is —(CH₂)ₙNH₂ or —(CH)ₙSH, where n=1-25;
a leaving group LG₁ is N-phenyltrifluoroacetimidate (CF₃C(═NPh)O—);
a leaving group LG₂ is selected from one of trichloroacetimidate (CCl₃C(═NH)O—), N-phenyltrifluoroacetimidate (CF₃C(═NPh)O—), methylthio (SMe), phenylselenyl (SePh), ethylthio (SEt), phenylthio (SPh), p-tolylthio (STol), and dibutylphosphono (—P(═O)—(OBu)₂);
the synthesis method includes the following steps:
(1) construction of disaccharide acceptor: removing a hydroxyl protecting group PG₆ at the 3 position of monosaccharide building block 8 to obtain acceptor 14; enabling the acceptor 14 and monosaccharide building block 7 to undergo a glycosylation reaction to obtain disaccharide 15; reducing an azide group in the disaccharide 15 to an amino group by a reducing agent, and adding compound 13 for amidation to obtain compound 16; removing a hydroxyl protecting group PG₄ at the 3 position of the monosaccharide building block 7 in compound 16 to obtain disaccharide acceptor 17;

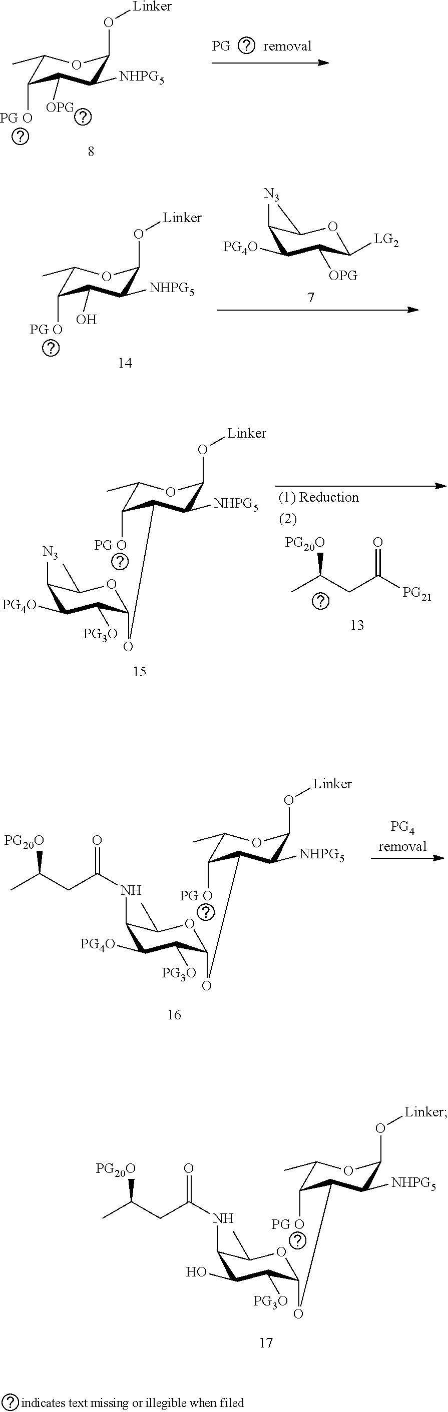

(2) construction of target trisaccharide:
enabling the disaccharide acceptor 17 and monosaccharide building block 6 to undergo a glycosylation reaction under the action of an activating agent to construct trisaccharide 18; reducing an azide group of the trisaccharide 18 by a reducing agent, and adding any one of the carboxylic acid derivatives represented by Formulae (9) to (12) for amidation to correspondingly obtain compounds 19 to 22; enabling the compounds 19 to 22 to undergo catalytic hydrogenation for deprotection to obtain target compounds 1 to 4;
or, enabling the trisaccharide 18 to undergo reductive acylation to convert an azide group to an acetylamino group, followed by catalytic hydrogenation for deprotection to obtain target compound 5;

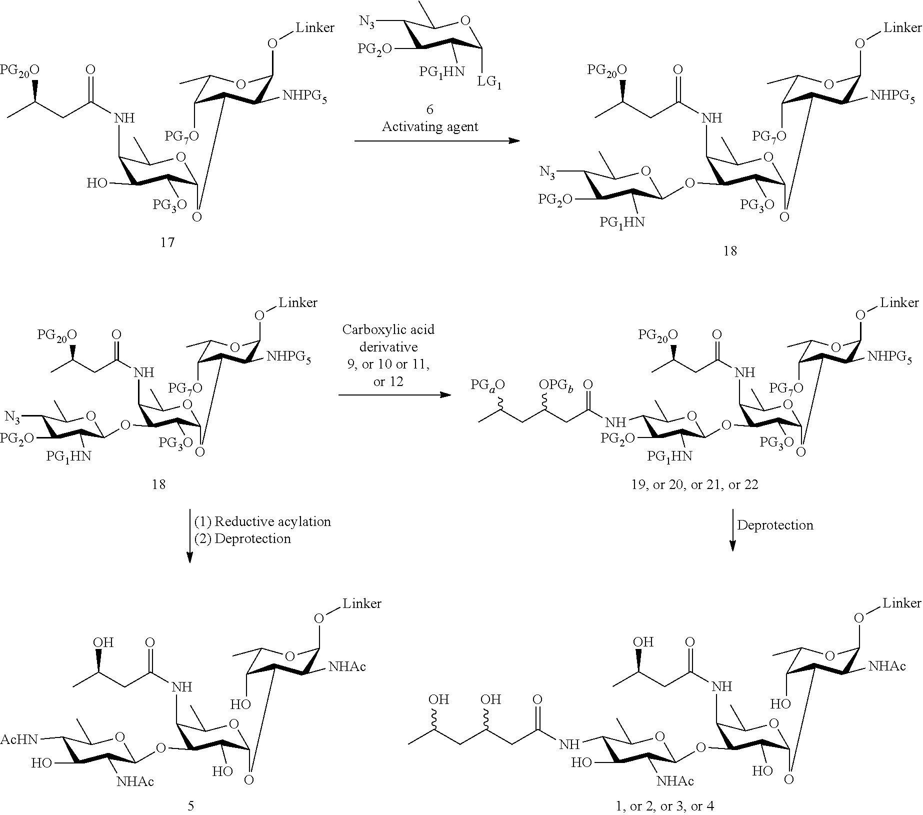

PGₐ and PG_{b} are temporary hydroxyl protecting groups each independently selected from benzyl, 2-naphthylmethyl, acetyl, benzoyl, neopentyl, 9-pentamethoxycarbonyl, and 2-p-methoxybenzyl.

In an implementation of the disclosure, C1-4 alkoxy includes methyl (Me), ethyl (Et), and tert-butyl (t-Bu). When the PG₁₀, PG₁₃, PG₁₆, PG₁₉, and PG₂₁ are C1-4 alkoxy, the protecting groups need to be removed before amide coupling.

In an implementation of the disclosure, the disaccharide acceptor 17 is synthesized by the way of: enabling the acceptor 14 and donor 7 to undergo a glycosylation reaction to obtain disaccharide 15 under the action of catalysis of an activating agent and a solvent effect; then, reducing an azide group in the disaccharide 15 to an amino group to obtain an amino disaccharide intermediate under the action of a reducing agent; and enabling (R)-3-hydroxybutyric acid derivative 13 to be activated by a condensing agent or prepared into acyl halide that is condensed with amino disaccharide intermediate amide to obtain compound 16, and removing a PG₄ protecting group in compound 16 to obtain the acceptor 17. A corresponding synthetic route is shown as follows:

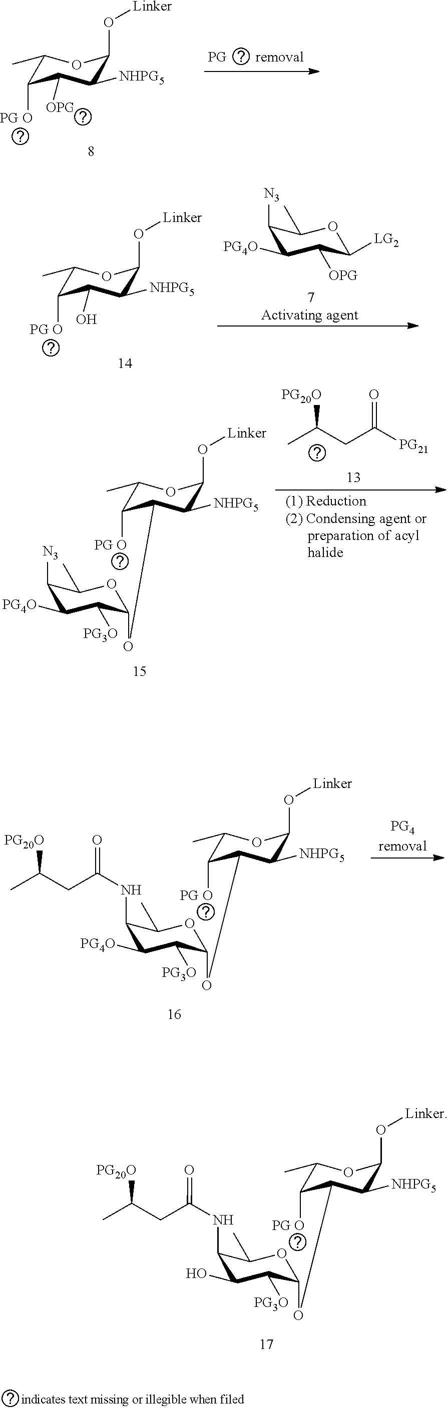

In an implementation of the disclosure, the disaccharide 15 is synthesized through the use of the solvent effect, catalysis of the activating agent, and orthogonal protection of non-participating groups.

In an implementation of the disclosure, the concentration of the glycosylation reaction is 0.01 mol/L-0.1 mol/L.

In an implementation of the disclosure, the activating agent is one of TMSOTf, NIS/TMSOTf, and NIS/TfOH.

In an implementation of the disclosure, the solvent is one or more of anhydrous dichloromethane, diethyl ether, toluene, methanol, tetra hydrofuran, acetonitrile, N,N-dimethylformamide, or water, and preferably is a mixed system of anhydrous dichloromethane, diethyl ether, and toluene.

In an implementation of the disclosure, the molar ratio of the donor to the acceptor is (1-3):1 or 1:(1-3).

In an implementation of the disclosure, specific reaction conditions for synthesizing disaccharides are: a glycosyl donor and a glycosyl acceptor are dissolved in a mixed solvent of toluene, dichloromethane, and diethyl ether and stirred under the protection of argon, a molecular sieve is added, the reaction temperature is −20° C. to 0° C., 0.1-0.3 equivalents of the activating agent (relative to the molar equivalent of the donor) is added, and the reaction time is 2 hours-8 hours.

In an implementation of the disclosure, the reducing agent for reducing the azide group in the disaccharide 15 is one of zinc powder, triphenylphosphine, 1,3-propanedithiol, lithium aluminum hydride, trimethylphosphine, stannous chloride dihydrate, sodium borohydride, and sodium cyanoborohydride.

In an implementation of the disclosure, the condensing agent is one of DCC (dicyclohexylcarbodiimide), DIC (diisopropylcarbodiimide), EDC (1-(3-dimethylaminopropyl)-3-ethylcarbodiimide), DPPA (Diphenyl phosphoryl azide), DPPCl (diphenylphosphinyl chloride), DECP (diphenyl cyanophosphonate), HATU (O-(7-Azabenzotriazol-1-yl)-N,N,N′,N′-tetramethyluronium hexafluorophosphate), HBTU (O-Benzotriazol-1-yl-N,N,N′,N′-tetramethyluronium hexafluorophosphate), and HCTU (6-Chlorobenzotriazol-1-yl)-N,N,N′,N′-tetramethyluronium hexafluorophosphate).

In an implementation of the disclosure, the acyl halide includes one of acyl chloride, acyl bromide, and acyl fluoride.

In an implementation of the disclosure, a method for preparing acyl chloride includes one of synthesis of acyl chloride using thionyl chloride, synthesis of acyl chloride using oxalyl chloride, and preparation of acyl chloride using trichloro-s-triazine.

In an implementation of the disclosure, the trisaccharide 18 is synthesized by the way of: enabling the acceptor 17 and donor 6 to undergo a glycosylation reaction under the catalysis of an activating agent to construct the trisaccharide 18 with a single configuration by means of the temperature effect and the neighboring group participation effect of the C2 position. A corresponding synthetic route is shown as follows:

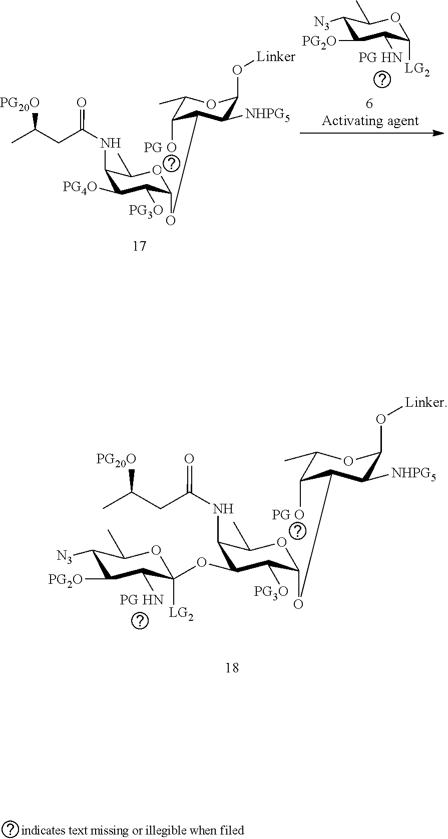

In an implementation of the disclosure, a 1,2-trans-β-glycosidic linkage in the trisaccharide 18 is constructed by means of the temperature effect, the catalysis of the activating agent, and the neighboring group participation effect of PG₁.

In an implementation of the disclosure, the concentration of the glycosylation reaction is 0.01-0.1 M.

In an implementation of the disclosure, the activating agent is one of TMSOTf, NIS/TMSOTf, and NIS/TfOH.

In an implementation of the disclosure, the solvent is one or more of anhydrous dichloromethane, diethyl ether, toluene, methanol, tetrahydrofuran, acetonitrile, N,N-dimethylformamide, or water.

In an implementation of the disclosure, the molar ratio of the donor to the acceptor is (1-3):1 or 1:(1-3).

In an implementation of the disclosure, glycosylation reaction conditions of the trisaccharide 18 include: the disaccharide acceptor 17 and the donor 6 are dissolved in a dichloromethane solvent, a molecular sieve is added, 0.2-1 equivalent of the activating agent (relative to the molar weight of the acceptor) is added, the temperature of the reaction is controlled to gradually rise from 0° C. to room temperature, and the reaction time is 2-8 h.

In an implementation of the disclosure, a synthesis method of compound 19 includes: reducing an azide group at the 4 position of the non-reducing end of the trisaccharide 18 by a reducing agent to obtain an amino trisaccharide intermediate; and enabling compound 9 to be activated by a condensing agent or prepared into acyl halide that is then coupled with amino trisaccharide intermediate amide to obtain compound 19. A corresponding synthetic route is shown as follows:

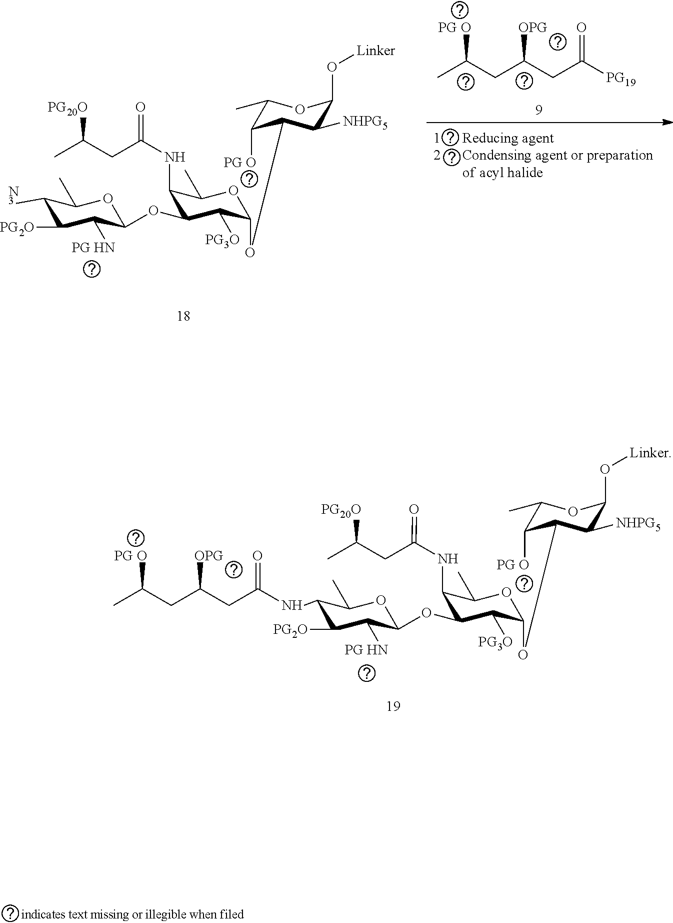

In an implementation of the disclosure, the reducing agent is one of zinc powder, triphenylphosphine, 1,3-propanedithiol, lithium aluminum hydride, trimethylphosphine, stannous chloride dihydrate, sodium borohydride, and sodium cyanoborohydride.

In an implementation of the disclosure, the condensing agent is one of DCC (dicyclohexylcarbodiimide), DIC (diisopropylcarbodiimide), EDC (1-(3-dimethylaminopropyl)-3-ethylcarbodiimide), DPPA (diphenylphosphoryl azide), DPPCl (diphenylphosphinyl chloride), DECP (diphenyl cyanophosphonate), HATU (2-(7-azabenzotriazole)-N,N,N′,N′-tetramethylurea hexafluorophosphate), HBTU (benzotriazole-N,N,N′,N′-tetramethylurea hexafluorophosphate), and HCTU (6-chlorobenzotriazole-1,1,3,3-tetramethylurea hexafluorophosphate).

In an implementation of the disclosure, the acyl halide includes one of acyl chloride, acyl bromide, and acyl fluoride.

In an implementation of the disclosure, a method for preparing acyl chloride includes one of synthesis of acyl chloride using thionyl chloride, synthesis of acyl chloride using oxalyl chloride, and preparation of acyl chloride using trichloro-s-triazine.

In an implementation of the disclosure, a synthesis method of compounds 20, 21, and 22 is the same as or similar to the synthesis method of compound 19. That is, compounds 20, 21, and 22 are synthesized by reducing the azide group at the 4 position of the non-reducing end of the trisaccharide 18 by the reducing agent to obtain the amino trisaccharide intermediate, and enabling compound 10 or 11 or 12 to be respectively coupled with amino trisaccharide intermediate amide.

In an implementation of the disclosure, compounds 1 to 5 are synthesized by the way of: enabling compounds 19, 20, 21, and 22 (PGₐ and PG_{b} represent corresponding hydroxyl protecting groups) to respectively undergo catalytic hydrogenation under palladium-on-carbon hydrogenation conditions for deprotection to obtain target compounds 1 to 4; and enabling the trisaccharide 18 to be reduced by a reducing agent and acetylated, converting the azide group at the 4 position of the non-reducing end 4 to an acetylamino group, and then, performing catalytic hydrogenation for deprotection to obtain the target compound 5. A corresponding synthetic route is shown as follows:

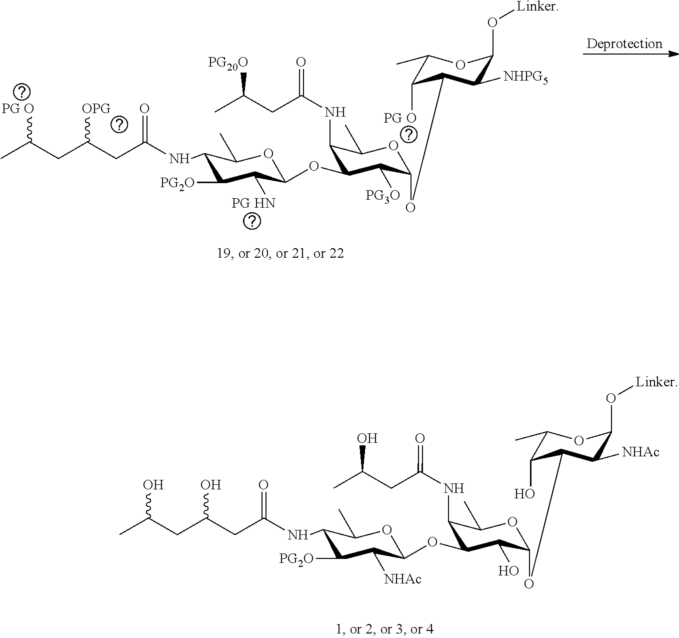

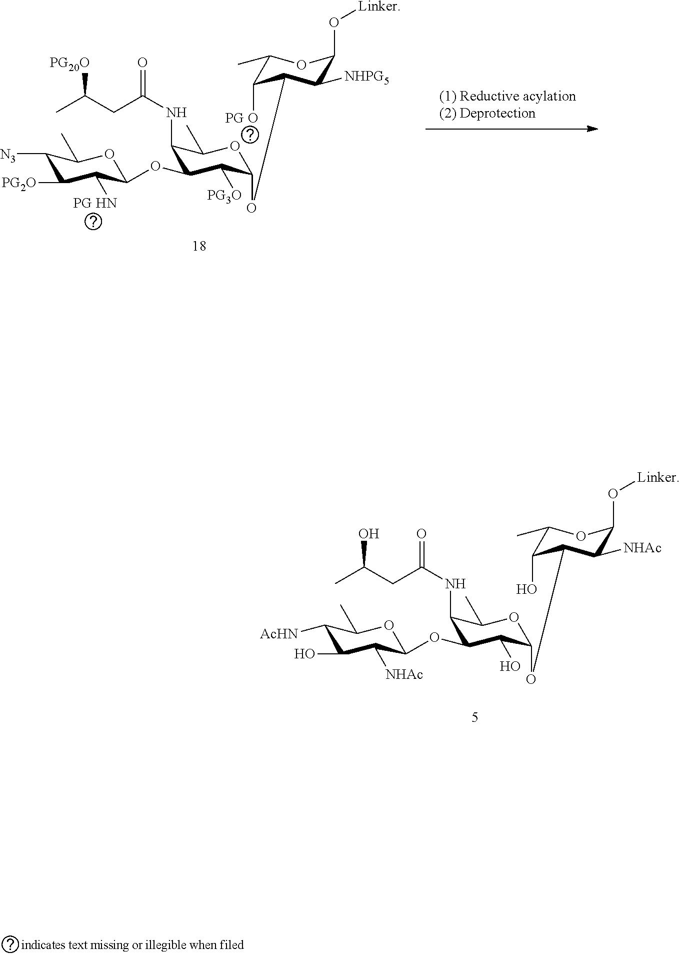

In an implementation of the disclosure, in the deprotection method, a method for removing ester protecting groups may be implemented through potassium hydroxide/methanol/water, sodium hydroxide/ethanol/water, lithium hydroxide/methanol/water, sodium hydroxide/methanol/water, potassium hydroxide/ethanol/water, lithium hydroxide/ethanol/water, etc.

In an implementation of the disclosure, in the deprotection method, a method for removing silicon ether protecting groups may be implemented through tetrabutylammonium fluoride, hydrofluoric acid, etc.

In an implementation of the disclosure, in the deprotection method, the removal of carbon ether protecting groups may be implemented through catalytic hydrogenation, that is, in the presence of a catalyst, hydrogen is introduced for reaction.

In an implementation of the disclosure, the catalyst used for the catalytic hydrogenation may be a 10% palladium-on-carbon catalyst or palladium hydroxide.

In an implementation of the disclosure, the solvent used for the deprotection reaction may be a mixed solution of water/methanol/dichloromethane/acetic acid, a mixed solution of water/tert-butyl alcohol/dichloromethane, a mixed solution of water/tert-butyl alcohol/tetrahydrofuran, etc., and the reaction temperature may be between 0 and 40° C.

In an implementation of the disclosure, in the synthesis of compound 5, the reducing agent is one of zinc powder, triphenylphosphine, 1,3-propanedithiol, lithium aluminum hydride, trimethylphosphine, stannous chloride dihydrate, sodium borohydride, and sodium cyanoborohydride.

In an implementation of the disclosure, in the synthesis of compound 5, the acetylation may be implemented through pyridine and acetic anhydride, or methanol and acetic anhydride, or acetic acid and acetic anhydride, etc.

In an implementation of the disclosure, the reductive acylation of compound 5 may also be implemented by directly adding compound 18 to a mixed solution of thioacetic acid and pyridine to react.

An application of the disclosure is to illustrate the absolute configuration of dHh by synthesized oligosaccharides.

In an implementation of the disclosure, the assignment of the absolute configuration of dHh is based on NMR error analysis using four synthesized oligosaccharide isomers (1-4) and extracted natural O-polysaccharides (OPS).

In an implementation of the disclosure, the OPS is extracted from V. cholerae serotype O100 inactivated bacteria.

In an implementation of the disclosure, in order to reduce the error as much as possible, nuclear magnetic testing instruments used for deprotection products (1-5) are all 600 M, and tests are performed at the same temperature (25° C.).

Another application of the disclosure is to illustrate the immunological effects of dHh by synthesized oligosaccharides.

In an implementation of the disclosure, the illustration of the immunological effects of dHh is implemented through a glycan microarray technology.

In an implementation of the disclosure, a preparation process of the glycan microarray includes: binding linkers at the reducing ends of five oligosaccharide fragments to a microarray, using antiserum for incubation, using secondary antibodies for labeling, and performing fluorescent scanning.

In an implementation of the disclosure, the antiserum is derived from animal serum immunized with V. cholerae serotype O100 lipopolysaccharides (LPS) or human serum infected with V. cholerae serotype O100.

In an implementation of the disclosure, glycan microarray results indicate that neither the absence nor the configuration change of dHh both affect its binding ability to antibodies.

The disclosure further provides a V. cholerae glycoconjugate for vaccine research and development. The conjugate is formed by covalently linking above-mentioned five oligosaccharide fragments (1-5) of compounds to proteins. The general formula of the conjugate may be expressed as Sugar-Linker-carrier protein.

In an implementation of the disclosure, the carrier protein includes one of diphtheria toxin mutant protein (CRM197), keyhole limpet hemocyanin (KLH), bovine serum albumin (BSA), meningococcal outer membrane protein complex (OMPC), tetanus toxoid (TT), or diphtheria toxoid (DT).

The disclosure further provides an application of the glycoconjugate in preparation of a vaccine. The vaccine is used for preventing or treating diseases caused by V. cholerae infection.

Further, the main symptoms of the diseases is one or more of diarrhea, vomiting, depressed eye socket, dry skin, disturbance of consciousness, shock, and death.

The disclosure further provides an application of the above-mentioned chemical synthesis method in preparation of a glycan microarray or V. cholerae glycoconjugate. The application includes the following processes:
S1: preparing V. cholerae serotype O100 O-antigen oligosaccharide fragments with linkers by the above-mentioned chemical synthesis method; and
S2: then binding the linkers of the obtained oligosaccharide fragments to a microarray or carrier protein to obtain a corresponding glycan microarray or V. cholerae glycoconjugate.

### Beneficial Effects

The disclosure develops an efficient and concise synthesis method of O-antigen oligosaccharides by three monosaccharide building blocks and five carboxylic acid derivatives by means of the neighboring group participation effect, remote participation effect, solvent effect, etc., through a series of orthogonal protection, stereoselective assembly and efficient amide coupling. Five oligosaccharide fragments are successfully synthesized by the method.

The absolute configurations (3S, 5S) of dHh are illustrated by four synthesized oligosaccharide isomers (1-4) in combination with the NMR technology. The determination of the absolute configuration lays a foundation for the exploration of the subsequent structure-activity relationship. The five oligosaccharide fragments (1-5) are prepared into the glycan microarray. Glycan microarray screening indicates that dHh is not an essential structural feature of the antigenic epitope. Next, other oligosaccharide fragments may be synthesized with reference to the method described in the disclosure, thereby screening the minimal antigenic epitope.

In summary, the disclosure will provide a reliable theoretical basis for V. cholerae vaccine design, infection diagnosis, and drug research and development.

### BRIEF DESCRIPTION OF FIGURES

FIG. 1 shows five oligosaccharide fragments and three monosaccharide building blocks and five carboxylic acid derivatives required;

FIG. 2 shows a synthetic route of four hexanoic acid derivatives 18* to 22*;

FIG. 3 shows a synthetic route of a disaccharide acceptor 28*;

FIG. 4 shows a synthetic route of trisaccharide 31*;

FIG. 5 shows a synthetic route of fully protected trisaccharides 32* to 35*;

FIG. 6 shows a synthetic route of target compounds (1* to 5*);

FIG. 7 shows a full spectrum error analysis diagram of ¹³C-NMR of target compounds (1* to 4*);

FIG. 8 shows a processed spectrum error analysis diagram of ¹³C-NMR of target compounds (1* to 4*), where 2′ to 6′ represent NMR signals of different ¹³C in dHh, and similarly, 2″ to 4″ represent NMR signals of different ¹³C in RHb;

FIG. 9A shows comparison diagrams of glycan microarray screening results, where FIG. 9A shows a schematic structural diagram of oligosaccharides, FIG. 9B shows a microarray printing pattern, FIG. 9C shows a microarray scanning result, FIG. 9D shows quantification of the mean fluorescence intensity, and error bars represent the standard error of the mean of two spots at uniform concentration;

FIG. 10 shows an NMR-HSQC structural identification diagram of compound 1*;

FIG. 11 shows an NMR-HSQC structural identification diagram of compound 2*;

FIG. 12 shows an NMR-HSQC structural identification diagram of compound 3*;

FIG. 13 shows an NMR-HSQC structural identification diagram of compound 4*; and

FIG. 14 shows an NMR-HSQC structural identification diagram of compound 5*.

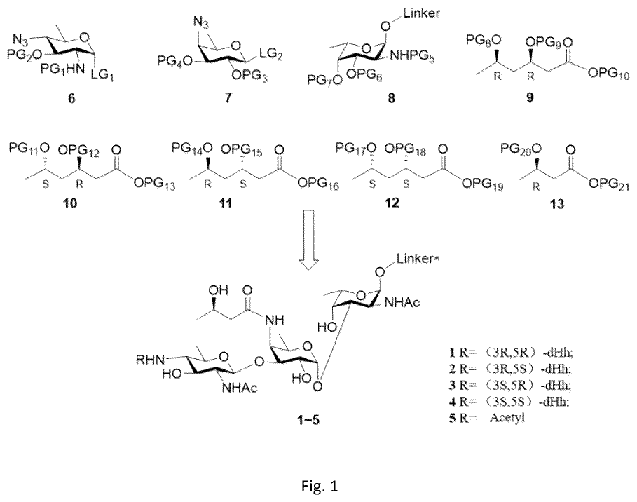

### DETAILED DESCRIPTION

Commercial agents used in experiments are used as received without further treatment, and anhydrous solvents used in reactions are prepared by an MBraun MB-SPS 800 type solvent drying system. Solvents used for silica gel column chromatography are all analytically pure and are used after reduced pressure distillation. A silica gel plate used for thin layer chromatography (TLC) is a glass based or aluminum foil based silica gel plate prepared from 60-F254 silica gel, and the silica gel used for normal-phase silica gel column chromatography is carried out with 200-300 mesh silica gel.

The yield of each reaction step is calculated as follows: (amount of target product substance/amount of raw material substance)*100%. The structures of products are identified by a nuclear magnetic resonance (NMR) spectrum, an infrared spectrum, optical rotation, and a high-resolution mass spectrum. The purity of products is analyzed by the NMR spectrum. A proton NMR spectrum, a carbon-13 NMR spectrum, and two-dimensional NMR spectrum are measured by Bruker Ascend 600 M and 400 M NMR spectrometers at 25° C. The high-resolution mass spectrum is measured by an Agilent 6220 electrospray ionization time-of-flight mass spectrometer. The infrared spectrum is measured by a Thermo Fisher Scientific Nicolet iS5 FT-IR spectrometer. The optical rotation is measured by a Schmidt & Haensch UniPol L 10000 fully-automatic polarimeter at 589 nm. The unit for measuring concentration (c) is g/100 mL.

### Example 1

### Synthesis of 3,5-Dibenzylhexanoic Acid

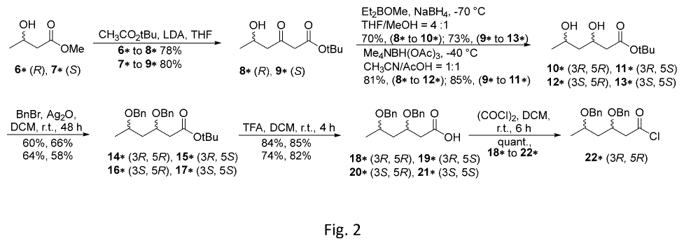

As shown in FIG. 2: (R)-3-hydroxybutyrate methyl ester 6* and (S)-3-hydroxybutyrate methyl ester 7* were used as starting materials respectively and subjected to Claisen condensation to obtain (R)-5-hydroxyhexanoate tert-butyl ester 8* and (S)-5-hydroxyhexanoate tert-butyl ester 9*. Four different configurations of tert-butyl 3,5-dihydroxyhexanoates 10* to 13* were selectively constructed further by Narasaka and Evans methods. Compounds 14* to 17* were obtained by selective benzylation mediated by silver oxide. Tert-butyl was removed by trifluoroacetic acid to obtain 3,5-dibenzylhexanoic acids 18* to 21*.

Specific experiment operations and steps:

Compound 8*: At −78° C., lithium diisopropylamide (1.25 mL, 2.5 mmol) and tert-butyl acetate (0.34 mL, 2.51 mmol) were added to a tetrahydrofuran (1 mL) solution and stirred for 20 min. Compound 6* (0.1 g, 0.85 mmol) was dissolved in a THF (15 mL) solution, and then, the solution was added dropwise to a reaction system. Stirring was performed at −50° C. for 2 h. After the reaction of raw materials was completed as detected by TLC, deionized water was added, extraction was performed with ethyl acetate, an organic phase was collected, dried over anhydrous sodium sulfate and filtered to remove sodium sulfate, concentration was performed by distillation under reduced pressure using a rotary evaporator, and a crude product was separated and purified by silica gel column chromatography (petroleum ether:ethyl acetate, 4:1, v/v) to obtain compound 8* (134 mg, 0.66 mmol, 78%). [α]²⁵D=−13.7° (c=0.2, CHCl₃); IR νmax (film) 2973, 2931, 1731, 1715, 1456, 1368, 1252, 1145, 959, 839, 754 cm-1; ¹H NMR (600 MHz, Chloroform-d) δ 4.26 (ddq, J=9.6, 6.4, 3.1 Hz, 1H, 5-H), 3.37 (d, J=2.2 Hz, 2H, 2-CH₂), 2.85 (d, J=3.4 Hz, 1H, 5-OH), 2.76-2.60 (m, 2H, 4-CH₂), 1.47 (s, 9H, tBu-CH₃), 1.21 (d, J=6.3 Hz, 3H, 6-CH₃). ¹³C NMR (151 MHz, Chloroform-d) δ 204.2, 166.1, 63.8, 51.1, 28.0, 22.4; HRMS (ESI) calculated for C₁₀H₁₈O₄Na⁺ [M+Na]⁺: 225.1097, found: 225.1083.

Compound 9*: At −78° C., lithium diisopropylamide (1.25 mL, 2.5 mmol) and tert-butyl acetate (0.34 mL, 2.51 mmol) were added to a tetrahydrofuran (1 mL) solution and stirred for 20 min. Compound 7* (0.1 g, 0.85 mmol) was dissolved in a THF (15 mL) solution, and then, the solution was added dropwise to a reaction system. Stirring was performed at −50° C. for 2 h. After the reaction of raw materials was completed as detected by TLC, deionized water was added, extraction was performed with ethyl acetate, an organic phase was collected, dried over anhydrous sodium sulfate and filtered to remove sodium sulfate, concentration was performed by distillation under reduced pressure using a rotary evaporator, and a crude product was separated and purified by silica gel column chromatography (petroleum ether:ethyl acetate, 4:1, v/v) to obtain compound 9* (137 mg, 0.68 mmol, 80%). [α]²⁵D=+9.9° (c=0.3, CHCl₃); IR νmax (film) 2971, 2925, 2853, 1731, 1714, 1456, 1368, 1257, 1145, 958, 939, 754 cm⁻¹; ¹H NMR (400 MHz, Chloroform-d) δ 4.27 (dtd, J=12.6, 6.3, 3.0 Hz, 1H, 5-H), 3.39 (d, J=1.1 Hz, 2H, 2-CH₂), 2.79-2.60 (m, 2H, 4-CH₂), 1.49 (s, 9H, tBu-CH₃), 1.23 (d, J=6.4 Hz, 3H, 6-CH₃). ¹³C NMR (101 MHz, Chloroform-d) δ 204.3, 166.1, 63.8, 51.1, 50.9, 28.0, 22.3; HRMS (ESI) calculated for C₁₀H₁₈O₄Na⁺ [M+Na]⁺: 225.1097, found: 225.1083.

Compound 10*: At −70° C., compound 8* (150 mg, 0.74 mmol) was dissolved in a mixed solution of tetrahydrofuran and methanol (4:1, v/v, 7.5 mL), and then, diethylmethoxyborane (0.8 mL, 0.8 mmol) was added. After one hour, sodium borohydride (31 mg, 0.82 mmol) was added to the solution. Then, at −70° C., the reaction was continued for 22 h. After the reaction of raw materials was completed as detected by TLC, a saturated ammonium chloride solution was added, and extraction was performed three times with ethyl acetate. An organic layer was washed with a saturated sodium bicarbonate solution and brine and then dried over anhydrous sodium sulfate, concentration was carried out under vacuum, and a product was purified by silica gel column chromatography (petroleum ether:ethyl acetate, 1:1, v/v) to obtain 10* (105.7 mg, 0.52 mmol, 70%). [α]²⁵D=−165.5° (c=0.1, CHCl₃); IR νmax (film) 3028, 2976, 2933, 1715, 1368, 1272, 1154, 1069, 843, 760, 605 cm⁻¹; ¹H NMR (600 MHz, Chloroform-d) δ 4.23 (dq, J=9.0, 5.6 Hz, 1H, 3-H), 4.07 (dqd, J=8.9, 6.2, 2.5 Hz, 1H, 5-H), 3.80 (s, 1H, 3-OH), 3.43 (s, 1H, 5-OH), 2.40 (d, J=6.1 Hz, 2H, 2-CH₂), 1.61-1.49 (m, 2H, 4-CH₂), 1.47 (s, 9H, tBu-CH₃), 1.20 (d, J=6.2 Hz, 3H, 6-CH₃). ¹³C NMR (151 MHz, Chloroform-d) δ 172.2, 81.6, 69.2, 68.2, 44.0, 42.6, 28.1, 23.7; HRMS (ESI) calculated for C₁₀H₂₀O₄Na⁺ [M+Na]⁺: 227.1254, found: 227.1283.

Compound 11*: At room temperature, triacetoxytetramethylborane (3.0 g, 11.5 mmol) was added to a mixed solution of dry acetonitrile (9.1 mL) and acetic acid (9.1 mL) and stirred for 30 min. Then, compound 9* (370 mg, 1.83 mmol) was dissolved in an acetonitrile (2.7 mL) solution, and the solution was added dropwise to a reaction system at −40° C. and stirred for 94 h. After the reaction of raw materials was completed as detected by TLC, quenching was performed with a saturated potassium sodium tartrate solution, and dilution and extraction were performed with dichloromethane. An organic layer was washed with a saturated sodium bicarbonate solution and then dried over anhydrous sodium sulfate, concentration was carried out under vacuum, and a product was purified by silica gel column chromatography (petroleum ether:ethyl acetate, 1:1, v/v) to obtain dihydroxy ester 11* (317 mg, 1.56 mmol, 85%). [α]²⁵D=+63.1° (c=0.9, CHCl₃); IR νmax (film) 2974, 2931, 1727, 1393, 1367, 1257, 1152, 1084, 954, 842, 760, 664 cm⁻¹; ¹H NMR (400 MHz, Chloroform-d) δ 4.33 (dq, J=8.4, 4.0 Hz, 1H, 3-H), 4.16 (d, J=7.8 Hz, 1H, 5-H), 3.58 (d, J=3.4 Hz, 1H, 3-OH), 2.60 (d, J=4.4 Hz, 1H, 5-OH), 2.54-2.37 (m, 2H, 2-CH₂), 1.65-1.55 (m, 2H, 4-CH₂), 1.49 (s, 9H, tBu-CH₃), 1.26 (d, J=6.3 Hz, 3H, 6-CH₃). ¹³C NMR (101 MHz, Chloroform-d) δ 172.5, 81.5, 65.9, 65.8, 65.1, 65.0, 43.5, 43.5, 42.2, 42.1, 42.0, 28.2, 28.1, 23.6, 23.5; HRMS (ESI) calculated for C₁₀H₂₀O₄Na⁺ [M+Na]⁺: 227.1254, found: 227.1271.

Compound 12*: At room temperature, triacetoxytetramethylborane (3.0 g, 11.5 mmol) was added to a mixed solution of dry acetonitrile (9.1 mL) and acetic acid (9.1 mL) and stirred for 30 min. Then, compound 8* (370 mg, 1.83 mmol) was dissolved in an acetonitrile (2.7 mL) solution, and the solution was added dropwise to a reaction system at −40° C. and stirred for 94 h. After the reaction of raw materials was completed as detected by TLC, quenching was performed with a saturated potassium sodium tartrate solution, and dilution and extraction were performed with dichloromethane. An organic layer was washed with a saturated sodium bicarbonate solution and then dried over anhydrous sodium sulfate, concentration was carried out under vacuum, and a product was purified by silica gel column chromatography (petroleum ether:ethyl acetate, 1:1, v/v) to obtain dihydroxy ester 12* (302 mg, 1.48 mmol, 81%). [α]²⁵D=+8.5° (c=0.3, CHCl₃); IR νmax (film) 2975, 2931, 1728, 1393, 1367, 1257, 1152, 1084, 954, 842, 760, 664 cm⁻¹; ¹H NMR (600 MHz, Chloroform-d) δ 4.31 (ddd, J=12.1, 8.4, 3.4 Hz, 1H, 3-H), 4.14 (ddp, J=9.4, 6.1, 3.1 Hz, 1H, 5-H), 3.57 (s, 1H, 3-OH), 2.59 (s, 1H, 5-OH), 2.51-2.33 (m, 2H, 2-CH₂), 1.65-1.53 (m, 2H, 4-CH₂), 1.47 (s, 9H, tBu-CH₃), 1.24 (d, J=6.3 Hz, 3H, 6-CH₃). ¹³C NMR (151 MHz, Chloroform-d) δ 172.5, 81.5, 65.9, 65.0, 43.5, 42.1, 28.1, 23.5; HRMS (ESI) calculated for C₁₀H₂₀O₄Na⁺ [M+Na]⁺: 227.1254, found: 227.1283.

Compound 13*: At −70° C., compound 9* (150 mg, 0.74 mmol) was dissolved in a mixed solution of tetrahydrofuran and methanol (4:1, v/v, 7.5 mL), and then, diethylmethoxyborane (0.8 mL, 0.8 mmol) was added. After one hour, sodium borohydride (31 mg, 0.82 mmol) was added to the solution. Then, at −70° C., the reaction was continued for 22 h. After the reaction of raw materials was completed as detected by TLC, a saturated ammonium chloride solution was added, and extraction was performed three times with ethyl acetate. An organic layer was washed with a saturated sodium bicarbonate solution and brine and then dried over anhydrous sodium sulfate, concentration was carried out under vacuum, and a product was purified by silica gel column chromatography (petroleum ether:ethyl acetate, 1:1, v/v) to obtain 13* (110.2 mg, 0.54 mmol, 73%). [α]²⁵D=+26.2° (c=0.2, CHCl₃); IR νmax (film) 2978, 2931, 1728, 1368, 1324, 1256, 1154, 1085, 843, 761, 664 cm⁻¹; ¹H NMR (600 MHz, Chloroform-d) δ 4.23 (dq, J=9.3, 6.4, 4.7 Hz, 1H, 3-H), 4.07 (dqd, J=8.9, 6.2, 2.3 Hz, 1H, 5-H), 3.79 (m, 1H, 3-OH), 3.42 (s, 1H, 5-OH), 2.40 (d, J=6.2 Hz, 2H, 2-CH₂), 1.59-1.51 (m, 2H, 4-CH₂), 1.47 (s, 9H, tBu-CH₃), 1.19 (s, 3H, 6-CH₃). ¹³C NMR (151 MHz, Chloroform-d) δ 172.2, 81.6 (1-C), 69.4, 69.2, 68.2, 44.0, 42.6, 28.1, 23.7. (ESI) calculated for C₁₀H₂₀O₄Na⁺ [M+Na]⁺: 227.1254, found: 227.1278.

Compound 14*: Under the protection of nitrogen, compound 10* (642.6 mg, 3.15 mmol) was dissolved in anhydrous dichloromethane (31.5 mL), and benzyl bromide (3.74 mL, 31.5 mmol) and silver oxide (8.99 g, 37.8 mmol) were added at 0° C. The reaction mixture was stirred at 0° C. for 5 h and then stirred at room temperature for 28 h. After filtration through diatomite and concentration, an organic layer was extracted with a saturated sodium bicarbonate solution and then dried over anhydrous sodium sulfate, concentration was carried out under vacuum, and purification was performed by silica gel column chromatography (petroleum ether:ethyl acetate, 9:1, v/v) to obtain product 14* (726 mg, 1.89 mmol, 60%). [α]²⁵D=−26.1° (c=1.1, CHCl₃); IR νmax (film) 3029, 2931, 1714, 1453, 1373, 1271, 1143, 1094, 1065, 746, 697 cm⁻¹; ¹H NMR (400 MHz, Chloroform-d) δ 7.33-7.11 (m, 10H,Ar), 4.49 (dd, J=11.5, 3.2 Hz, 2H, Ar—CH₂), 4.39 (dd, J=15.7, 11.5 Hz, 2H, Ar—CH₂), 3.94 (ddd, J=12.6, 6.9, 5.6 Hz, 1H, 3-H), 3.59 (h, J=6.2 Hz, 1H, 5-H), 2.44 (dd, J=15.0, 7.2 Hz, 1H, 2-CH₂), 2.34 (dd, J=15.0, 5.4 Hz, 1H, 2-CH₂), 1.98 (dt, J=13.6, 6.7 Hz, 1H, 4-CH₂), 1.58-1.52 (m, 1H, 4-CH₄), 1.37 (s, 9H, tBu-CH₃), 1.13 (d, J=6.1 Hz, 3H, 6-CH₃). ¹³C NMR (101 MHz, Chloroform-d) δ 171.1, 138.9, 138.7, 128.5, 128.4, 127.9, 127.7, 127.7, 127.6, 80.7, 73.9, 71.9, 71.5, 70.4, 41.6, 41.3, 28.3, 19.8; HRMS (ESI) calculated for C₂₄H₃₂O₄Na⁺ [M+Na]⁺: 407.2193, found: 407.2232.

Compound 15*: Under the protection of nitrogen, compound 11* (764 mg, 3.65 mmol) was dissolved in anhydrous dichloromethane (36.5 mL), and benzyl bromide (4.34 mL, 36.52 mmol) and silver oxide (10.4 g, 43.8 mmol) were added at 0° C. The reaction mixture was stirred at 0° C. for 5 h and then stirred at room temperature for 28 h. After filtration through diatomite and concentration, an organic layer was extracted with a saturated sodium bicarbonate solution and then dried over anhydrous sodium sulfate, concentration was carried out under vacuum, and purification was performed by silica gel column chromatography (petroleum ether:ethyl acetate, 9:1, v/v) to obtain product 15* (926 mg, 2.41 mmol, 66%). [α]²⁵D=+44.6° (c=0.68, CHCl₃); IR νmax (film) 2974, 2930, 1728, 1454, 1367, 1256, 1152, 1065, 954, 843, 736, 697 cm⁻¹; ¹H NMR (400 MHz, Chloroform-d) δ 7.35-7.26 (m, 10H, Ar), 4.60 (dd, J=11.4, 3.5 Hz, 2H, Ar—CH₂), 4.36 (dd, J=18.1, 11.4 Hz, 2H, Ar—CH₂), 4.13 (m, J=6.2 Hz, 1H, 3-H), 3.78 (m, J=6.1 Hz, 1H, 5-H), 2.60-2.42 (m, 2H, 2-CH₂), 1.77-1.72 (m, 2H, 4-CH₂), 1.47 (s, 9H, tBu-CH₃), 1.23 (d, J=6.1 Hz, 3H, 6-CH₃). ¹³C NMR (101 MHz, Chloroform-d) δ 170.9, 138.6, 138.6, 128.3, 127.8, 127.7, 127.5, 127.4, 80.5, 73.4, 71.9, 71.6, 71.5, 70.3, 43.3, 41.7, 28.1, 28.1, 28.1, 20.0, 19.9. HRMS (ESI) calculated for C₂₄H₃₂O₄Na⁺ [M+Na]⁺: 407.2193, found: 407.2233.

Compound 16*: Under the protection of nitrogen, compound 12* (950 mg, 4.65 mmol) was dissolved in anhydrous dichloromethane (46.5 mL), and benzyl bromide (5.5 mL, 46.5 mmol) and silver oxide (13.3 g, 55.8 mmol) were added at 0° C. The reaction mixture was stirred at 0° C. for 5 h and then stirred at room temperature for 28 h. After filtration through diatomite and concentration, an organic layer was extracted with a saturated sodium bicarbonate solution and then dried over anhydrous sodium sulfate, concentration was carried out under vacuum, and purification was performed by silica gel column chromatography (petroleum ether:ethyl acetate, 9:1, v/v) to obtain product 16* (1.14 g, 0.025 mol, 64%). [α]²⁵D=−48.8° (c=0.4, CHCl₃); IR νmax (film) 2931, 1730, 1455, 1368, 1257, 1156, 1116, 1063, 846, 735, 697 cm⁻¹; ¹H NMR (400 MHz, Chloroform-d) δ 7.35-7.20 (m, 10H, Ar), 4.58 (dd, J=11.4, 3.3 Hz, 2H, Ar—CH₂), 4.34 (dd, J=17.9, 11.4 Hz, 2H, Ar—CH₂), 4.11 (m, J=6.2 Hz, 1H, 3-H), 3.76 (m, J=6.2 Hz, 1H, 5-H), 2.54 (dd, J=14.8, 6.4 Hz, 1H, 2-CH₂), 2.43 (dd, J=14.8, 5.8 Hz, 1H, 2-CH₂), 1.76-1.68 (m, 2H, 4-CH₂), 1.44 (s, 9H, tBu-CH₃), 1.21 (d, J=6.1 Hz, 3H, 6-CH₃). ¹³C NMR (101 MHz, Chloroform-d) δ 170.9, 139.0, 138.6, 128.3, 127.8, 127.7, 127.5, 127.4, 80.5, 73.5, 71.9, 71.6, 71.5, 70.3, 43.3, 41.7, 28.1, 19.9. HRMS (ESI) calculated for C₂₄H₃₂O₄Na⁺ [M+Na]⁺: 407.2193, found: 407.2250.

Compound 17*: Compound 13* (319 mg, 1.56 mmol) was dissolved in anhydrous dichloromethane (15.6 mL), and benzyl bromide (1.85 mL, 15.6 mmol) and silver oxide (4.45 g, 18.74 mmol) were added at 0° C. The reaction mixture was stirred at 0° C. for 5 h and then stirred at room temperature for 28 h. After filtration through diatomite and concentration, an organic layer was extracted with a saturated sodium bicarbonate solution and then dried over anhydrous sodium sulfate, concentration was carried out under vacuum, and purification was performed by silica gel column chromatography (petroleum ether:ethyl acetate, 9:1, v/v) to obtain product 17* (347.6 mg, 0.905 mmol, 58%). [α]²⁵D=−4.7° (c=0.2, CHCl₃); IR νmax (film) 2927, 1729, 1454, 1368, 1258, 1157, 1090, 1064, 844, 760, 697 cm⁻¹; ¹H NMR (600 MHz, Chloroform-d) δ 7.36-7.23 (m, 10H, Ar), 4.56 (dd, J=11.5, 4.2 Hz, 2H, Ar—CH₂), 4.46 (dd, J=23.3, 11.5 Hz, 2H, Ar—CH₂), 4.01 (m, J=6.3 Hz, 1H, 3-H), 3.66 (m, J=6.2 Hz, 1H, 5-H), 2.51 (dd, J=15.0, 7.2 Hz, 1H, 2-CH₂), 2.41 (dd, J=15.0, 5.4 Hz, 1H, 2-CH₂), 2.05 (dt, J=13.7, 6.7 Hz, 1H, 4-CH₂), 1.67-1.60 (m, 1H, 4-CH₂), 1.45 (s, 9H, tBu-CH₃), 1.20 (d, J=6.1 Hz, 3H, 6-CH₃). ¹³C NMR (151 MHz, Chloroform-d) δ 170.9, 138.8, 138.5, 128.4, 128.3, 127.8, 127.7, 127.5, 127.5, 80.5, 73.8, 71.7, 71.3, 70.2, 41.4, 41.1, 28.1, 19.7. HRMS (ESI) calculated for C₂₄H₃₂O₄Na⁺ [M+Na]⁺: 407.2193, found: 407.2221.

Compound 18*: Under the protection of nitrogen, tert-butyl 3,5-dibenzylhexanoate 14* (61 mg, 0.159 mmol) was dissolved in anhydrous DCM (1 mL), and then, trifluoroacetic acid (1 mL) was added to a reaction system at 0° C. The temperature was slowly raised to room temperature, and the reaction was continued for 12 h. After the reaction of raw materials was completed as detected by TLC, dichloromethane was added for dilution, an organic layer was extracted with ultrapure water and a saturated sodium bicarbonate solution respectively and then dried over anhydrous sodium sulfate, concentration was carried out under vacuum, and a crude product was purified by silica gel column chromatography (petroleum ether:ethyl acetate=1:1) to obtain compound 18* (43.8 mg, 0.134 mmol, 84%). [α]²⁵D=−10.8° (c=1.0, CHCl₃); IR νmax (film) 2963, 2928, 1727, 1454, 1367, 1270, 1154, 1119, 1069, 794, 696 cm⁻¹; ¹H NMR (400 MHz, Chloroform-d) δ 7.42-7.13 (m, 10H, Ar), 4.64-4.48 (m, 3H, Ar—CH₂), 4.41 (d, J=11.7 Hz, 1H, Ar—CH₂), 4.05 (p, J=6.2 Hz, 1H, 3-H), 3.73-3.56 (m, 1H, 5-H), 2.58 (dd, J=6.1, 1.4 Hz, 2H, 2-CH₂), 2.06 (ddd, J=13.7, 7.6, 5.7 Hz, 1H, 4-CH₂), 1.67 (ddd, J=14.3, 6.8, 5.0 Hz, 1H, 4-CH₂), 1.21 (d, J=6.1 Hz, 3H, 6-CH₃). ¹³C NMR (101 MHz, Chloroform-d) δ 176.2, 138.6, 138.1, 128.6, 128.0, 127.9, 127.9, 127.8, 127.7, 73.2, 71.6, 71.5, 70.6, 70.4, 41.1, 39.5, 21.5, 19.8. HRMS (ESI) calculated for C₂₀H₂₄O₄Na⁺ [M+Na]⁺: 351.1567, found: 351.1604.

Compound 19*: Under the protection of nitrogen, tert-butyl 3,5-dibenzylhexanoate 15* (74 mg, 0.19 mmol) was dissolved in anhydrous DCM (1 mL), and then, trifluoroacetic acid (1 mL) was added to a reaction system at 0° C. The temperature was slowly raised to room temperature, and the reaction was continued for 12 h. After the reaction of raw materials was completed as detected by TLC, dichloromethane was added for dilution, an organic layer was extracted with ultrapure water and a saturated sodium bicarbonate solution respectively and then dried over anhydrous sodium sulfate, concentration was carried out under vacuum, and a crude product was purified by silica gel column chromatography (petroleum ether:ethyl acetate=1:1) to obtain compound 19* (53.8 mg, 0.164 mmol, 85%). [α]²⁵D=+1020 (c=0.5, CHCl₃); IR νmax (film) 2927, 1715, 1451, 1274, 1176, 1111, 1069, 1026, 751, 712 cm⁻¹; ¹H NMR (600 MHz, Chloroform-d) δ 7.36-7.23 (m, 10H, Ar), 4.58 (dd, J=11.4, 9.0 Hz, 2H, Ar—CH₂), 4.38 (d, J=11.2 Hz, 1H, Ar—CH₂), 4.31 (d, J=11.5 Hz, 1H, Ar—CH₂), 4.19-4.04 (m, 1H, 3-H), 3.94-3.68 (m, 1H, 5-H), 2.70-2.55 (m, 2H, 2-CH₂), 1.84-1.65 (m, 2H, 4-CH₂), 1.22 (d, J=6.1 Hz, 3H, 6-CH₃). ¹³C NMR (151 MHz, Chloroform-d) δ 138.64-, 137.9, 128.5, 128.4, 128.0, 127.9, 127.85, 127.59, 73.0, 72.0, 71.4, 71.37, 70.3, 42.9, 39.6, 19.8; HRMS (ESI) calculated for C₂₀H₂₄O₄Na⁺ [M+Na]⁺: 351.1567, found, 351.1589.

Compound 20*: Under the protection of nitrogen, tert-butyl 3,5-dibenzylhexanoate 16* (100 mg, 0.26 mmol) was dissolved in anhydrous DCM (1 mL), and then, trifluoroacetic acid (1 mL) was added to a reaction system at 0° C. The temperature was slowly raised to room temperature, and the reaction was continued for 12 h. After the reaction of raw materials was completed as detected by TLC, dichloromethane was added for dilution, an organic layer was extracted with ultrapure water and a saturated sodium bicarbonate solution respectively and then dried over anhydrous sodium sulfate, concentration was carried out under vacuum, and a crude product was purified by silica gel column chromatography (petroleum ether:ethyl acetate=1:1) to obtain compound 20* (62 mg, 0.19 mmol, 74%). [α]²⁵D=−63.3° (c=0.75, CHCl₃); IR νmax (film) 3029, 2966, 2930, 1709, 1454, 1373, 1146, 1109, 1062, 736, 697 cm⁻¹; ¹H NMR (400 MHz, Chloroform-d) δ 7.39-7.24 (m, 10H, Ar), 4.61 (dd, J=11.4, 5.8 Hz, 2H, Ar—CH₂), 4.40 (d, J=11.2 Hz, 1H, Ar—CH₂), 4.34 (d, J=11.6 Hz, 1H, Ar—CH₂), 4.21-4.08 (m, 1H, 3-H), 3.87-3.74 (m, 1H, 5-H), 2.65 (d, J=5.8 Hz, 2H, 2-CH₂), 1.84-1.76 (m, 2H, 3-CH₂), 1.25 (d, J=6.1 Hz, 3H, 6-CH₃). ¹³C NMR (101 MHz, Chloroform-d) δ 176.4, 138.7, 138.0, 128.4, 128.42, 127.99, 127.87, 127.79, 127.61, 73.0, 72.0, 71.4, 70.3, 43.0, 39.8, 19.8. HRMS (ESI) calculated for C₂₀H₂₄O₄Na⁺ [M+Na]⁺: 351.1567, found, 351.1607.

Compound 21*: Under the protection of nitrogen, tert-butyl 3,5-dibenzylhexanoate 17* (89 mg, 0.23 mmol) was dissolved in anhydrous DCM (1 mL), and then, trifluoroacetic acid (1 mL) was added to a reaction system at 0° C. The temperature was slowly raised to room temperature, and the reaction was continued for 12 h. After the reaction of raw materials was completed as detected by TLC, dichloromethane was added for dilution, an organic layer was extracted with ultrapure water and a saturated sodium bicarbonate solution respectively and then dried over anhydrous sodium sulfate, concentration was carried out under vacuum, and a crude product was purified by silica gel column chromatography (petroleum ether:ethyl acetate=1:1) to obtain compound 21* (62.4 mg, 0.19 mmol, 82%). [α]²⁵D=+28.6° (c=0.4, CHCl₃); IR νmax (film) 3029, 2926, 2857, 1714, 1496, 1454, 1373, 1184, 1091, 1061, 749, 697, 608 cm⁻¹; ¹H NMR (400 MHz, Chloroform-d) δ 7.36-7.26 (m, 10H, Ar), 4.64-4.51 (m, 3H, Ar—CH₂), 4.41 (d, J=11.6 Hz, 1H, Ar—CH₂), 4.05 (p, J=6.1 Hz, 1H, 3-H), 3.68 (q, J=6.2 Hz, 1H, 5-H), 2.59 (d, J=6.0 Hz, 2H, 2-CH₂), 2.07 (ddd, J=13.7, 7.9, 5.4 Hz, 1H, 4-CH₂), 1.68 (ddd, J=14.2, 7.0, 4.7 Hz, 1H, 4-CH₂), 1.22 (d, J=6.1 Hz, 3H, 6-CH₃). ¹³C NMR (101 MHz, Chloroform-d) δ 174.5, 138.4, 137.8, 128.5, 127.9, 127.8, 127.7, 71.4, 70.3, 40.8, 39.1, 19.7, 19.7. HRMS (ESI) calculated for C₂₀H₂₄O₄Na⁺ [M+Na]⁺: 351.1567, found: 351.1588.

Compound 22*: Under the protection of nitrogen, tert-butyl 3,5-dibenzylhexanoate 18* (89 mg, 0.23 mmol) was dissolved in anhydrous DCM (1 mL), then oxalyl chloride was added to a reaction system at 0° C., and a catalytic amount of DMF was added to react at 0° C. for 30 min. After the reaction was completed as detected by TLC, quenching was performed with an appropriate amount of methanol to quantitatively obtain acyl chloride compound 22*. Due to the reactivity and easy hydrolysis of acyl chloride, the acyl chloride was prepared and used as needed in experiments.

### Example 2

### Synthesis of Disaccharide Acceptor 28*

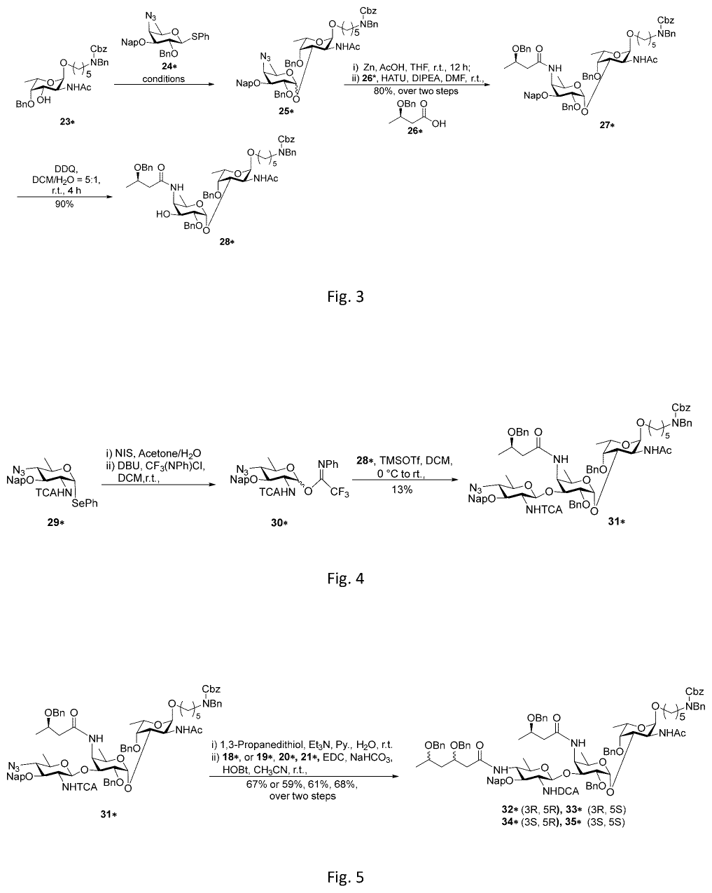

As shown in FIG. 3: L-fucosamine building block 23* and D-fucose building block 24* (Cai Juntao, doctoral thesis, Jiangnan University, 2020) were used as raw materials for a glycosylation reaction to obtain compound 25*. An azide group at the non-reducing end of disaccharide 25* was reduced to an amino group (that is an amino disaccharide amide) by preactivated zinc powder, and then, under the action of a condensing agent HATU (2-(7-azabenzotriazole)-N,N,N′,N′-tetramethylurea hexafluorophosphate), (R)-3-O-benzylbutyric acid 26* (which can be prepared referring to literature: Tanasova. et al, Angew. Chem. Int. Ed. 2015, 54 (14), 4274-4278) was condensed with the amino disaccharide to successfully introduce butanoyl at the C4′ position of disaccharide to obtain disaccharide 27*. Nap in disaccharide 27* was selectively removed by DDQ to obtain compound 28*.

### Experiment Operations and Steps:

Compound 25*: The synthesis of disaccharide 25* was initially attempted via a preactivation strategy, which failed to yield the desired product. Then, a donor and an acceptor were dissolved in a dichloromethane solution to undergo a glycosylation reaction under the catalysis of trimethylsilyl trifluoromethanesulfonate and N-iodosuccinimide to successfully obtain compound 25*, but the selectivity of the reaction was poor (α:β=1.5:1). Then, glycosylation reaction conditions were optimized again by the solvent effect of diethyl ether, so as to finally obtain compound 25* (α:β=6:1) with good selectively. A specific glycosylation method was as follows:

Method 1: Under the protection of argon, glycosyl donor 24* (53.2 mg, 0.104 mmol) was dissolved in anhydrous dichloromethane (2.0 mL) and cooled to −60° C. Then, a preactivated 4 Å molecular sieve, 1-(phenylsulfonyl)piperidine (BSP, 26.1 mg, 0.125 mmol), 2,4,6-tri-tert-butylpyrimidine (TTBP, 38.8 mg, 0.156 mmol), and trifluoromethanesulfonic anhydride (Tf2O, 22.7 μL, 0.135 mmol) were added and stirred at −60° C. to react for 30 min. TLC monitoring was performed to confirm the complete reaction of raw materials, then, 1-octene (16.3 μL, 0.104 mmol) was added and stirring was continued at −60° C. for reaction an additional 15 minutes. Then, the reaction solution was cooled to −78° C., glycosyl acceptor 23* (72.4 mg, 0.120 mmol) was dissolved in anhydrous dichloromethane (2.0 mL) and added dropwise to the reaction solution, and stirring was continued at −78° C. for reaction for an additional 3 h. After the reaction of raw materials was completed as confirmed by TLC monitoring, triethyl phosphite (53.5 μL, 0.312 mmol) was added to terminate the reaction, and then, stirring was continued at −78° C. for an additional 1 h. Through TLC detection of the reaction, it was found that the components of the reaction system were complex, and target compounds were not detected by liquid chromatography-mass spectrometry.

Method 2: D-fucose donor 24* (80.6 mg, 0.158 mmol) and L-fucose acceptor 23* (115 mg, 0.19 mmol) were dissolved in toluene (5 mL), azeotropically dehydrated three times, concentrated under vacuum and dried by rotary evaporation, then, a preactivated 4 Å molecular sieve was added and evacuation was performed on an oil pump overnight. Under the protection of argon, anhydrous dichloromethane (6.0 mL) and N-iodosuccinimide (43 mg, 0.19 mmol) were added sequentially, cooled to 0° C. and stirred for 30 min, and trimethylsilyl trifluoromethanesulfonate (5.7 μL, 0.032 mmol) was added dropwise and stirred at 0° C. to react for 5 h. After the reaction of raw materials was completed as detected by TLC, triethylamine was added to quench the reaction, diatomite was added to a fritted glass funnel, the molecular sieve was filtered out, extraction was performed with dichloromethane, washing was performed with a saturated sodium bicarbonate solution, an organic phase was collected and dried over anhydrous sodium sulfate, sodium sulfate was filtered out with filter paper, concentration was performed by distillation under reduced pressure using a rotary evaporator, and a crude product was separated and purified by silica gel column chromatography (petroleum ether:ethyl acetate, 1:1, v/v) to obtain a configuration disaccharide 25* (128.7 mg, 0.128 mmol, 81%, α:β=1.5:1).

Method 3: D-fucose donor 24* (100 mg, 0.196 mmol) and L-fucose acceptor 23* (141.6 mg, 0.235 mmol) were dissolved in toluene (5 mL), azeotropically dehydrated three times, concentrated under vacuum and dried by rotary evaporation, and then, a preactivated 4 Å molecular sieve was added and evacuation was performed on an oil pump overnight. Under the protection of argon, anhydrous dichloromethane (4.5 mL), diethyl ether (3.0 mL), and N-iodosuccinimide (52.9 mg, 0.235 mmol) were added sequentially, cooled to 0° C. and stirred for 30 min, and trimethylsilyl trifluoromethanesulfonate (3.45 μL, 0.039 mmol) was added dropwise and stirred at 0° C. to react for 5 h. After the reaction of raw materials was completed as detected by TLC, triethylamine was added to quench the reaction, diatomite was added to a fritted glass funnel, the molecular sieve was filtered out, extraction was performed with dichloromethane, washing was performed with a saturated sodium bicarbonate solution, an organic phase was collected and dried over anhydrous sodium sulfate, sodium sulfate was filtered out with filter paper, concentration was performed by distillation under reduced pressure using a rotary evaporator, and a crude product was separated and purified by silica gel column chromatography (petroleum ether:ethyl acetate, 1:1, v/v) to obtain α configuration disaccharide 25* (177 mg, 0.176 mmol, 90%, α:β=6:1, α configuration: ³J_{H1/H2}=3.4 Hz, ¹J_{H1/C1}=166 Hz). [α]²⁵D=+11.67° (c=1.0, CHCl₃); IR νmax (film) 3366, 2934, 2108, 1732, 1700, 1538, 1498, 1456, 1423, 1362, 1278, 1228, 1177, 1127, 1092, 1047, 858, 823, 752, 735, 700 cm⁻¹; a: ¹H NMR (400 MHz, CDCl3) δ=7.96-7.01 (m, 27H, Ar), 6.61 (d, J=5.7 Hz, 1H, NHAc), 5.15 (d, J=6.2 Hz, 2H, ArCH₂), 5.12 (d, J=3.5 Hz, 1H, 1-H), 4.95-4.78 (m, 5H, 1′-H, ArCH₂), 4.71 (dd, J=11.3, 5.1 Hz, 1H, ArCH₂), 4.63 (d, J=11.8 Hz, 1H, ArCH₂), 4.47 (d, J=6.9 Hz, 2H, ArCH₂), 4.32 (s, 1H, 2-H), 4.16-4.05 (m, 2H, 3′-H, 5′-H), 3.98 (dd, J=9.8, 3.4 Hz, 1H, 2′-H), 3.91 (t, J=11.6 Hz, 1H, 3-H), 3.85-3.74 (m, 2H, 4′-H, 5-H), 3.64-3.46 (m, 2H, 4-H, linker-OCH₂), 3.45-3.14 (m, 3H, linker-OCH₂, linker-NCH₂), 1.55 (ddt, J=17.5, 13.8, 6.5 Hz, 4H, linker-CH₂), 1.39 (d, J=4.5 Hz, 3H, Ac), 1.35-1.25 (m, 2H, linker-CH₂), 1.13 (d, J=6.4 Hz, 3H, 6′-CH₃), 1.09 (m, 3H, 6-CH₃); ¹³C NMR (100 MHz, CDCl3) δ=170.5, 156.8, 156.3, 138.6, 137.9, 137.3, 136.9, 136.8, 135.2, 133.3, 133.2, 129.1, 128.8, 128.7, 128.6, 128.4, 128.1, 128.0, 127.9, 127.7, 127.4, 127.3, 126.7, 126.5, 126.3, 125.7, 99.0, 97.0, 79.1, 78.9, 77.7, 75.3, 74.6, 73.3, 68.1, 67.3, 66.4, 66.2, 64.5, 50.5, 50.2, 50.0, 47.2, 46.2, 29.5, 28.1, 27.6, 23.6, 22.5, 17.5, 16.9. HR-ESI-MS (m/z): calcd for C₅₉H₆₇O₁₀N₅Na⁺(M+Na)+: 1028.4780 found: 1028.4786.

Compound 27*: Under the protection of nitrogen, disaccharide 25* (20.5 mg, 20.4 μmol) was dissolved in a mixed solution of tetrahydrofuran (0.8 mL) and acetic acid (0.2 mL), then preactivated zinc powder (0.5 g) was added, and the solution was stirred at room temperature for 12 h. Then, the reaction mixture was filtered and concentrated to obtain a crude product amino trisaccharide, and subsequently, the next reaction was performed directly. (R)-3-O-benzylbutyric acid 26* (6 mg, 30.6 μmol) and amino trisaccharide were dissolved in a DMF (1 mL) solution at room temperature, and HATU (9.3 mg, 22.48 μmol) and DIPEA (6.8 μL, 40.8 μmol) were added and stirred at room temperature for 6 h. After the reaction of raw materials was completed as detected by TLC, a crude product was dissolved in ethyl acetate and washed with a saturated sodium chloride solution, a separated organic layer was dried over anhydrous sodium sulfate, concentration was carried out under vacuum, and purification was performed by column chromatography (petroleum ether:ethyl acetate, 1:1, v/v) to obtain compound 27* (18.8 mg, 16.3 μmol, 80%). [α]²⁵D=+34.6° (c=1.2, CHCl₃); IR νmax (film) 3029, 2924, 2853, 1681, 1543, 1507, 1455, 1361, 1217, 1092, 1045, 758, 698 cm⁻¹; ¹H NMR (400 MHz, CDCl3) δ=7.96-7.01 (m, 32H, Ar), 6.80 (d, J=10.2 Hz, 1H, NHRHb), 6.60 (d, J=5.5 Hz, 1H, NHAc), 5.15 (s, 2H, ArCH₂), 5.10 (d, J=3.5 Hz, 1H, 1-H), 5.01 (d, J=11.1 Hz, 1H, ArCH₂), 4.86 (d, J=11.7 Hz, 1H, ArCH₂), 4.71-4.58 (m, 5H, 1′-H, 4′-H, ArCH₂), 4.55-4.43 (m, 5H, ArCH₂), 4.25 (m, 2H, 2-H, 5′-H), 4.01 (dt, J=6.4, 3.2 Hz, 1H, RHb-CH), 3.94 (dd, J=10.1, 4.2 Hz, 1H, 3′-H), 3.89-3.74 (m, 2H, 3-H, 5-H), 3.60-3.44 (m, 2H, 4-H, linker-OCH₂), 3.36-3.14 (m, 3H, linker-OCH₂, linker-NCH₂), 2.65 (dd, J=15.2, 3.7 Hz, 1H, RHb-CH₂), 2.52 (dd, J=15.2, 6.7 Hz, 1H, RHb-CH₂), 1.62-1.46 (m, 4H, linker-CH₂), 1.35-1.25 (m, 8H, RHb-CH₃, Ac, linker-CH₂), 1.10 (d, J=6.4 Hz, 3H, 6-CH₃), 1.03 (d, J=6.4 Hz, 3H, 6′-CH₃); ¹³C NMR (100 MHz, CDCl3) δ=171.7, 170.5, 138.7, 138.3, 138.0, 137.5, 135.7, 133.4, 133.24, 133.15, 132.3, 132.3, 132.2, 132.1, 132.0, 128.8, 128.69, 128.67, 128.6, 128.54, 128.48, 128.4, 128.2, 128.1, 128.03, 128.00, 127.94, 127.89, 127.8, 127.7, 127.4, 127.1, 126.4, 126.2, 126.1, 98.8, 97.0, 78.9, 77.8, 75.2, 74.5, 72.9, 71.6, 70.9, 68.1, 67.3, 66.8, 66.3, 50.2, 50.0, 43.6, 29.5, 23.5, 22.4, 19.1, 17.0, 16.9. HR-ESI-MS (m/z): calcd for C70H81012N3Na⁺(M+Na)+: 1178.5712 found: 1178.5704.

Compound 28*: Under the protection of argon, compound 27* (26.8 mg, 23.0 μmol) was dissolved in dichloromethane (5.0 mL), and deionized water (1.0 mL) and 2,3-dichloro-5,6-dicyano-1,4-benzoquinone (DDQ) (7.7 mg, 35.0 μmol) were added and stirred at room temperature to react for 5 h. After the reaction of raw materials was completed as monitored by TLC, extraction was performed with dichloromethane, washing was performed with a 10% (w/w) sodium thiosulfate solution, an organic phase was collected and dried over anhydrous sodium sulfate, sodium sulfate was filtered out with filter paper, concentration was performed by distillation under reduced pressure using a rotary evaporator, and a crude product was separated and purified by silica gel column chromatography (petroleum ether:ethyl acetate, 1:1, v/v) to obtain compound 28* (21 mg, 20.7 μmol, 90%). [α]²⁵D=−14.4° (c=1.0, CHCl₃); IR νmax (film) 3342, 3030, 2936, 1670, 1541, 1496, 1453, 1422, 1362, 1304, 1216, 1180, 1092, 1043, 827, 754, 698 cm⁻¹; ¹H NMR (400 MHz, CDCl3) δ=7.42-7.13 (m, 25H, Ar), 6.81 (d, J=8.6 Hz, 1H, NHRHb), 6.61 (d, J=5.8 Hz, 1H, NHAc), 5.15 (d, J=4.9 Hz, 2H, ArCH₂), 5.08 (d, J=3.5 Hz, 1H, 1-H), 4.84 (d, J=11.8 Hz, 1H, ArCH₂), 4.77 (d, J=11.0 Hz, 1H, ArCH₂), 4.67-4.53 (m, 4H, 1′-H, ArCH₂), 4.51-4.43 (m, 3H, ArCH₂), 4.35-4.20 (m, 3H, 2-H, 4′-H, 5′-H), 4.14 (m, 1H, 3′-H), 4.00 (dt, J=6.2, 3.5 Hz, 1H, RHb-CH), 3.86 (d, J=11.2 Hz, 1H, 3-H), 3.80 (d, J=7.3 Hz, 1H, 5-H), 3.60-3.44 (m, 2H, 4-H, linker-OCH₂), 3.38 (dd, J=9.8, 3.5 Hz, 1H, 2′-H), 3.36-3.18 (m, 3H, linker-OCH₂, linker-NCH₂), 2.63 (dd, J=15.0, 3.8 Hz, 1H, RHb-CH₂), 2.50 (dd, J=15.2, 6.6 Hz, 1H, RHb-CH₂), 1.57 (s, 3H, Ac), 1.56-1.46 (m, 4H, linker-CH₂), 1.37 (d, J=6.2 Hz, 3H, RHb-CH₃), 1.33-1.24 (m, 2H, linker-CH₂), 1.11 (d, J=6.4 Hz, 3H, 6-CH₃), 0.95 (d, J=6.4 Hz, 3H, 6′-CH₃); ¹³C NMR (100 MHz, CDCl3) δ=173.8, 170.7, 138.7, 138.04, 137.97, 137.8, 136.9, 128.71, 128.67, 128.6, 128.44, 128.36, 128.1, 127.94, 127.90, 127.88, 127.7, 127.4, 127.3, 98.6, 97.1, 79.2, 78.6, 74.9, 74.5, 72.5, 71.7, 70.9, 68.0, 67.3, 66.3, 66.1, 54.7, 49.9, 47.2, 46.3, 43.5, 29.8, 29.4, 28.1, 27.6, 23.5, 22.8, 19.1, 17.0, 16.7. HR-ESI-MS (m/z): calcd for C59H73O12N3Na⁺(M+Na)+: 1038.5086 found: 1038.5117.

### Example 3

### Synthesis of Trisaccharide 31*

As shown in FIG. 4: Known D-quinovosamine building block 29* (which can be prepared referring to existing literature Codée. et al, Organic & Biomolecular Chemistry. 2020,18 (15), 2834-2837) was dissolved in a mixed solution of acetone and H₂O and hydrolyzed under the catalysis of N-iodosuccinimide (NIS). Then, under the action of 2,2,2-trifluoro-N-phenylacetimidoyl chloride (171 μL, 1.14 mmol) and 1,8-diazabicycloundec-7-ene (DBU), trifluoroacetimidate 30* was obtained. Trifluoroacetimidate 30* and disaccharide acceptor 28* were subjected to a glycosylation reaction under the condition of catalysis of trimethylsilyl trifluoromethanesulfonate (TMSOTf) to successfully obtain single 6 configuration trisaccharide compound 31* (yield: 13%).

Due to the relatively low synthesis yield of compound 31*, glycosylation conditions were further optimized in the disclosure. After the glycosyl donor was replaced from compound 30* with compound 29*, a glycosylation reaction was performed under the condition of catalysis of trimethylsilyl trifluoromethanesulfonate and N-iodosuccinimide to obtain trisaccharide 31* with a yield of 28%. Subsequently, compound 29* was used as the glycosyl donor, and the glycosylation yield was successfully increased to 54% by changing the equivalent of the catalyst. Then, the glycosylation yield was further optimized by changing the temperature, and it was found that a better glycosylation yield may be obtained by slowly raising the temperature from 0° C. to room temperature. Finally, under the dual promotion of the temperature effect and the catalyst, the target trisaccharide was successfully obtained with a relatively high yield of 81%. Specific optimization processes and reaction conditions were shown in Table 1:

TABLE 1

| | | | | Reaction | | Yield (31*, β |
|---|---|---|---|---|---|---|
| Number | Donor | Acceptor | Catalyst | temperature | | configuration) |
| 1 | 30* | 28* | 0.2 eq TMSOTf | 0° | C. | 13% |
| 2 | 29* | 28* | 0.2 eq TMSOTf NIS | 0° | C. | 28% |
| 3 | 29* | 28* | 0.1 eq TMSOTf, NIS | 0° | C. | 8% |
| 4 | 29* | 28* | 0.4 eq TMSOTf, NIS | 0° | C. | 54% |
| 5 | 29* | 28* | 0.6 eq TMSOTf, NIS | 0° | C. | 49% |
| 6 | 29* | 28* | 0.4 eq TMSOTf, NIS | −20° | C. | 35% |

| | | | | | |
|---|---|---|---|---|---|
| 7 | 29* | 28* | 0.4 eq TMSOTf, NIS | 0° C. to r.t., | 81% |

### Specific Experiment Operations and Steps:

Compound 30*: Compound 29* (232.5 mg, 0.38 mmol) was dissolved in acetone and H₂O (10:1, v/v, 5.5 mL) at room temperature and stirred uniformly, and then, NIS (171.4 mg, 0.7 mmol) was added and stirred for 1 h. After the reaction was completed as displayed by TLC, the mixture was diluted with ethyl acetate and washed with 10% (w/v) Na₂S₂O₃. An organic layer was dried over Na₂SO₄ and filtered, and concentration was carried out under vacuum. The residue was purified by silica gel column chromatography (petroleum ether/ethyl acetate, 20/1-1/1, v/v) to obtain an intermediate compound. The intermediate compound was dissolved in a DCM (4.8 mL) solution at 0° C., and then, 2,2,2-trifluoro-N-phenylacetimidoyl chloride (171 μL, 1.14 mmol) and 1,8-diazabicycloundec-7-ene (DBU) (171 μL, 1.14 mmol) were added. The solution was stirred at 0° C. to react for 3 h, the mixture was concentrated under vacuum, and then, purification was performed by silica gel column chromatography (petroleum ether/ethyl acetate, 30/1-10/1, v/v) to obtain trifluoroacetimidate 30* (223.4 mg, 0.34 mmol, two-step yield: 90%). ¹H NMR (400 MHz, Chloroform-d) δ=7.96-7.76 (m, 5H), 7.59-7.33 (m, 6H), 7.30-7.05 (m, 1H), 6.27 (d, J=7.4, 1H), 4.94 (q, J=11.9, 2H), 4.38 (dd, J=7.4, 4.1, 1H), 3.85 (dd, J=5.7, 4.1, 1H), 3.50 (dq, J=9.6, 6.1, 1H), 3.34 (dd, J=9.7, 5.7, 1H), 1.38 (d, J=6.1, 3H). ¹³C NMR (101 MHz, Chloroform-d) δ=162.9, 135.1, 134.2, 133.2, 133.2, 129.4, 128.5, 128.0, 127.7, 127.6 126.4, 126.3, 126.2, 126.0, 105.6, 78.9, 72.3, 67.9, 66.7, 64.6, 19.1, HR-ESI-MS (m/z): calcd for C28H25Cl3F3N5O₄Na⁺(M+Na)+: 680.0924, found: 680.1030

Compound 31*: Two representative glycosylation reaction operation methods were as follows:

Method 1: Under the protection of argon, trifluoroacetimidate 30* (24.3 mg, 37 μmol) and disaccharide acceptor 28* (25.3 mg, 25 umol) were dissolved in toluene, azeotropically dehydrated and dried by rotary evaporation three times, evacuation was performed on an oil pump for 2 h, then a preactivated 4 Å molecular sieve and anhydrous dichloromethane (1.0 mL) were added, the reaction solution was cooled to 0° C. and stirred for 30 min, trimethylsilyl trifluoromethanesulfonate (0.9 μL, 5.0 μmol) was added dropwise, and the solution was stirred at 0° C. to react for 5 h. After the reaction of raw materials was completed as detected by TLC, triethylamine was added to quench the reaction, the molecular sieve was filtered out with diatomite, extraction was performed with dichloromethane, washing was performed with a saturated sodium bicarbonate solution, an organic phase was collected and dried over anhydrous sodium sulfate, concentration was performed by distillation under reduced pressure using a rotary evaporator, and a crude product was separated and purified by silica gel column chromatography (petroleum ether:ethyl acetate, 4:1, v/v) to obtain syrup 31* (4.78 mg, 3.25 μmol, 13%).

Method 2: Under the protection of argon, selenoglycoside 29* (110 mg, 0.18 mmol) and disaccharide acceptor 28* (122 mg, 0.12 mmol) were dissolved in toluene, azeotropically dehydrated and dried by rotary evaporation three times, evacuation was performed on an oil pump for 2 h, Then a preactivated 4 Å molecular sieve and anhydrous dichloromethane (6.0 mL) were added, the reaction solution was cooled to 0° C. and stirred for 30 min, trimethylsilyl trifluoromethanesulfonate (8.7 μL, 0.048 mmol) and N-iodosuccinimide (40.5 mg, 0.18 mmol) were added dropwise, the temperature was slowly raised from 0° C. to room temperature, and the solution was stirred to react for 5 h. After the reaction of raw materials was completed as detected by TLC, triethylamine was added to quench the reaction, the molecular sieve was filtered out with diatomite, extraction was performed with dichloromethane, washing was performed with a saturated sodium bicarbonate solution, an organic phase was collected and dried over anhydrous sodium sulfate, concentration was performed by distillation under reduced pressure using a rotary evaporator, and a crude product was separated and purified by silica gel column chromatography (petroleum ether:ethyl acetate, 4:1, v/v) to obtain syrup 31* (142.8 mg, 97.2 μmol, 81%). [α]²⁵D=+15.2° (c=0.7, CHCl₃); IR νmax (film) 3343, 3029, 2979, 2940, 2877, 2108, 1659, 1532, 1496, 1454, 1423, 1360, 1309, 1216, 1167, 1092, 1047, 822, 756, 698 cm⁻¹; ¹H NMR (600 MHz, Methanol-d₄) δ 7.85-7.72 (m, 4H, Ar), 7.47-7.14 (m, 28H, Ar), 5.13 (d, J=12.9 Hz, 2H, Ar—CH₂), 5.06-4.96 (m, 1H, 1-H′), 4.94-4.86 (m, 3H, Ar—CH₂), 4.82 (d, J=8.0 Hz, 2H, 1-H, 1″-H), 4.69-4.63 (m, 2H, Ar—CH₂), 4.60 (d, J=11.3 Hz, 2H, Ar—CH₂), 4.51-4.47 (m, 3H, Ar—CH₂), 4.45-4.38 (m, 2H, 4′-H, 2-H), 4.24-4.15 (m, 2H, 3′-H, 5′-H), 4.10-4.05 (m, 1H, RHb-3), 3.99 (m, 1H, 3-H), 3.87 (m, 2H, 2″-H, 5-H), 3.74 (m, 2H, 2′-H, 3″-H), 3.66 (d, J=9.1 Hz, 1H, 4-H), 3.54 (s, 1H, Linker-OCH₂), 3.33 (m, 1H, Linker-OCH₂), 3.28-3.18 (m, 3H, 4″-H, Linker-NCH₂), 3.11-3.03 (m, 1H, 5″-H), 2.60-2.49 (m, 2H, RHb-2), 1.66 (d, J=10.2 Hz, 3H, NHAc—CH₃), 1.51 (s, 4H, Linker-CH₂), 1.33 (d, J=6.2 Hz, 3H, RHb-4), 1.29-1.24 (m, 2H, Linker-CH₂), 1.18 (d, J=6.1 Hz, 6H, 6″-CH₃, 6-CH₃), 0.96 (d, J=6.4 Hz, 3H, 6′-CH₃). ¹³C NMR (151 MHz, Methanol-d₄) δ 173.1 (NH—C═O), 162.4 (NH—C═O), 138.8 (Ar), 138.6 (Ar), 138.1 (Ar), 135.2 (Ar), 133.3 (Ar), 133.1 (Ar), 128.3 (Ar), 126.2 (Ar), 125.7 (Ar), 125.5 (Ar), 100.2 (1″-H), 98.1 (1′-H), 97.0 (1-H), 80.0 (3″-C), 78.5 (4-C), 76.4 (2′-C), 75.3 (3-C), 75.1 (Ar—CH₂), 74.0 (3′-C), 72.9 (Ar—CH₂, RHb-3), 70.6 (5″-C), 70.4 (Ar—CH₂), 67.6 (4″-C), 67.4 (Ar—CH₂), 66.6 (5-C, Ar—CH₂), 66.2 (5′-C), 58.0 (2″-C), 53.8 (4′-C), 50.1 (2-C, Ar—CH₂), 46.6 (Linker-NCH₂) 43.2 (RHb-2), 28.8 (Linker-CH₂), 23.04 (Linker-CH₂), 21.6 (NHAc—CH₃), 18.9 (RHb-4), 17.2 (6″-CH₃), 15.7 (6′-CH₃, 6-CH₃). HR-ESI-MS (m/z): calcd for C78H90Cl3N7O15Na⁺(M+Na)+: 1492.5453 found: 1492.5469.

### Example 4

### Synthesis of Trisaccharides 32*, 33*, 34*, and 35*

As shown in FIG. 5: An azide group at the non-reducing end in trisaccharide 31* was reduced by mild 1,3-propanedithiol to obtain amino trisaccharide. Then, in the presence of 1-ethyl-(3-(dimethylamino)propyl)carbodiimide and 1-hydroxybenzotriazole, amino trisaccharide and 3,5-dibenzylhexanoic acid 18*, 19*, 20*, or 21* were subjected to an amide condensation reaction respectively to obtain fully protected trisaccharides 32* to 35*. The above-mentioned amide condensation method was optimized through a series of trial and error methods.

Compound 31* was used as a raw material. First, it was attempted to reduce the azide group with triphenylphosphine and use newly prepared acyl chloride 22* for amide coupling, but only a trace amount of target compound 32* was obtained. Subsequently, it was attempted to combine 1,3-propanedithiol with HATU to obtain compound 32* with a yield of 18%. Then, reduction conditions of the azide group were optimized continuously, and it was found that in the presence of pyridine, water, and triethylamine, the reduction yield can be better increased by 1,3-propanedithiol. The condensing agent was further optimized, and EDC was ultimately determined to be a better condensing agent. In general, the combined use of 1,3-propanedithiol and EDC can significantly increase the total yield of amide condensation. Specific optimization processes and reaction conditions were shown in Table 2:

TABLE 2

| | | Condensing | | |
|---|---|---|---|---|
| | | agent or | | Yield (32*, |
| Number | Reducing agent | acyl halide | Specific reaction condition | two-step) |
| 1 | Triphenylphosphine | Acyl | i)31*, PPh₃, THF, 40° C., 4 h; | Trace |
| | | chloride | H₂O, 65° C., 24 h; | |
| | | | ii)22*, Et₃N, DCM, r.t., | |
| 2 | 1,3-Propanedithiol | HATU | i)31*, 1,3-Propanedithiol, Et₃N, | 18% |
| | | | DMF, r.t., | |
| | | | ii)18*, HATU, DIPEA, DMF, r.t., | |
| 3 | Zinc powder | HATU | i)31*, Zn, AcOH, THF, r.t., 12 h; | Trace |
| | | | ii)18*, HATU, DIPEA, DMF, r.t., | |
| 4 | Triphenylphosphine | HATU | i)31*, PPh₃, THF/H₂O = 9:1 50° C., 3 h; | No product |
| | | | ii)18*, HATU, DIPEA, DMF, r.t., | was detected |
| 5 | 1,3-Propanedithiol | HATU | i) 31*, 1,3-Propanedithiol, Et₃N, Py., | 31% |
| | | | H₂O, r.t. | |
| | | | ii) 18*, HATU, DIPEA, DMF, r.t., | |
| 6 | 1,3-Propanedithiol | EDC | i) 31*, 1,3-Propanedithiol, Et₃N, Py., | 67% |
| | | | H₂O, r.t. | |
| | | | ii) 18*, EDC, HOBt, NaHCO₃, CHCN, | |
| | | | r.t., | |
| 7 | 1,3-Propanedithiol | DPPA | i) 31*, 1,3-Propanedithiol, Et₃N, Py., | Trace |
| | | | H₂O, r.t. | |
| | | | ii) 18*, DPPA, Et₃N, DMF, r.t. | |

### Specific Experiment Operations and Steps:

Compound 32*: Under the protection of nitrogen, trisaccharide 31* (30 mg, 20.4 μmol) was dissolved in a mixed solution of water (1 mL), Et₃N (124.8 μl, 0.90 mmol) and pyridine (4 mL), and then, 1,3-propanedithiol (122 μl, 1.22 mmol) was added and stirred at room temperature for 6 h. Then, the reaction mixture was concentrated to obtain a crude product amino trisaccharide, and the next reaction was performed directly. 3,5-dibenzylhexanoic acid 18* (10 mg, 30.6 μmol) and amino trisaccharide were dissolved in an acetonitrile (5 mL) solution at room temperature, and sodium bicarbonate (5.1 mg, 61.2 μmol) was added. After 10 min, 1-hydroxybenzotriazole (0.55 mg, 4.08 μmol) and 1-ethyl-(3-(dimethylamino)propyl)carbodiimide (6.25 mg, 32.6 μmol) were added sequentially and stirred at room temperature for 6 h. After the reaction of raw materials was completed as detected by TLC, a crude product was dissolved in ethyl acetate and washed with a saturated sodium chloride solution, a separated organic layer was dried over anhydrous sodium sulfate, concentration was carried out under vacuum, and purification was performed by column chromatography (DCM:MeOH=20:1) to obtain compound 32* (20.5 mg, 13.67 μmol, 67%). [α]²⁵D=+7.2° (c=0.36, CHCl₃); IR νmax (film) 2925, 2857, 1748, 1660, 1543, 1496, 1454, 1374, 1240, 1081, 1046, 811, 757, 698 cm⁻¹; ¹H NMR (600 MHz, Methanol-d₄) δ 7.80-7.67 (m, 4H, Ar), 7.46-7.11 (m, 39H, Ar), 5.9 (s, 1H, DCA-CH), 5.1 (d, J=11.8 Hz, 2H, Ar—CH₂), 4.97 (s, 1H, 1′-H), 4.90 (d, J=11.8 Hz, 1H, Ar—CH₂), 4.85 (d, J=8.3 Hz, 2H, 1-H, 1″-H), 4.75 (d, J=11.3 Hz, 1H, Ar—CH₂), 4.72-4.64 (m, 3H, Ar—CH₂), 4.59 (d, J=11.4 Hz, 2H, Ar—CH₂), 4.51-4.39 (m, 6H, Ar—CH₂, 4′-H, 2-H), 4.37-4.31 (m, 2H, Ar—CH₂), 4.28 (d, J=11.6 Hz, 1H, Ar—CH₂), 4.16 (dd, J=10.2, 4.9 Hz, 2H, 3′-H, 5′-H), 4.09-4.03 (m, 1H, RHb-3), 4.02-3.96 (m, 1H, 3-H), 3.89 (dt, J=12.3, 5.4 Hz, 3H, dHh-3, 4-H, 5-H), 3.77 (dd, J=10.2, 3.8 Hz, 2H, 2″-H, 2-H), 3.69 (m, 1H, 4″-H), 3.64 (m, 1H, 3″-H), 3.58 (h, J=6.0 Hz, 2H, dHh-5-H, Linker-OCH₂), 3.40 (m, 1H, 5″-H)), 3.34 (m, 1H, Linker-OCH₂) 3.23 (s, 2H, Linker-NCH₂), 2.61-2.49 (m, 2H, RHb-2), 2.33-2.21 (m, 2H, dHh-2), 1.90 (dt, J=13.6, 6.7 Hz, 1H, dHh-4), 1.72 (d, J=9.4 Hz, 3H, NHAc—CH₃), 1.53 (dt, J=14.0, 5.8 Hz, 5H, dHh-4, Linker-CH₂), 1.35 (d, J=6.1 Hz, 3H, RHb-4), 1.30-1.26 (m, 2H, Linker-CH₂), 1.17 (d, J=6.4 Hz, 3H, 6-CH₃), 1.08 (dd, J=6.1, 3.5 Hz, 6H, 6″-CH₃, dHh-4), 0.95 (d, J=6.5 Hz, 3H, 6′-CH₃). ¹³C NMR (151 MHz, Methanol-d₄) δ=172.9 (NH—C═O), 172.1 (NH—C═O), 164.7 (NH—C═O), 138.8 (Ar), 138.7 (Ar), 138.6 (Ar), 138.3 (Ar), 138.2 (Ar), 135.9 (Ar), 133.3 (Ar), 133.0 (Ar), 128.3 (Ar), 127.8 (Ar), 127.7-127.5 (Ar), 127.4 (Ar), 127.7 (Ar), 127.1 (Ar), 125.9 (Ar), 125.6 (Ar), 125.4 (Ar), 100.7 (1″-C), 98.2 (1′-C), 97.1 (1-C), 78.5 (3″-C), 76.2 (3-C), 74.9 (Ar—CH₂), 74.8 (3′-C), 73.4 (dHh-3), 72.9 (Ar—CH₂, RHb-3), 72.4 (Ar—CH₂), 71.8 (dHh-5), 70.5 (Ar-CH₂, 5″-C), 69.8 (Ar—CH₂), 67.5 (Linker-OCH₂), 67.0 (Ar—CH₂), 66.6 (Ar—CH₂), 66.5 (5′-C), 66.4 (DCA-CH₂), 57.1 (2″-C, 4″-C), 53.6 (4′-C), 50.1 (2-C, Ar—CH₂), 46.5 (Linker-NCH₂) 43.1 (RHb-2), 40.9 (dHh-4, dHh-2), 31.4 (1-C), 28.8 (Linker-CH₂), 27.1 (Linker-CH₂), 23.2 (1-C), 22.3 (Linker-CH₂), 21.8 (NHAc—CH₃), 18.8 (RHb-4), 18.5 (dHh-6), 17.0 (6″-C), 15.7 (6-C), 15.7 (6′-C). HR-ESI-MS (m/z): calcd for C98H115Cl2N5O18Na⁺(M+Na)+: 1742.7506 found: 1742.7550.

Compound 33*: Under the protection of nitrogen, trisaccharide 31* (30 mg, 20.4 μmol) was dissolved in a mixed solution of water (1 mL), Et₃N (124.8 μl, 0.90 mmol) and pyridine (4 mL), and then, 1,3-propanedithiol (122 μl, 1.22 mmol) was added and stirred at room temperature for 6 h. Then, the reaction mixture was concentrated to obtain a crude product amino trisaccharide, and the next reaction was performed directly. 3,5-dibenzylhexanoic acid 19* (10 mg, 30.6 μmol) and amino trisaccharide were dissolved in an acetonitrile (5 mL) solution at room temperature, and sodium bicarbonate (5.1 mg, 61.2 μmol) was added. After 10 min, 1-hydroxybenzotriazole (0.55 mg, 4.08 μmol) and 1-ethyl-(3-(dimethylamino)propyl)carbodiimide (6.25 mg, 32.6 μmol) were added sequentially and stirred at room temperature for 6 h. After the reaction of raw materials was completed as detected by TLC, a crude product was dissolved in ethyl acetate and washed with a saturated sodium chloride solution, a separated organic layer was dried over anhydrous sodium sulfate, concentration was carried out under vacuum, and purification was performed by column chromatography (DCM:MeOH=20:1) to obtain compound 33* (20.7 mg, 12.04 μmol, 59%). [α]²⁵D=+11.6° (c=0.42, CHCl₃); IR νmax (film) 2926, 2857, 1749, 1660, 1543, 1499, 1454, 1374, 1240, 1081, 1046, 819, 757, 698 cm⁻¹; ¹H NMR (600 MHz, Methanol-d₄) δ 7.80-7.67 (m, 4H, Ar), 7.50-7.08 (m, 38H, Ar), 5.95 (s, 1H, DCA-H), 5.13 (d, J=10.8 Hz, 2H, Ar—CH₂), 5.02-4.84 (m, 4H, 1′-H, Ar—CH₂, 1-H, 1″-H), 4.75-4.64 (m, 4H, Ar—CH₂), 4.62-4.54 (m, 2H, Ar—CH₂), 4.49-4.38 (m, 7H, Ar—CH₂, 4″-H, 2-H), 4.25-4.12 (m, 4H, 3′-H, 5′-H, Ar—CH₂), 4.07-3.86 (m, 5H, RHb-3, dHh-3, 3-H, 4-H, 5-H), 3.83-3.74 (m, 2H, 2″-H, 2′-H), 3.72 (s, 1H, 3″-H), 3.64 (dtd, J=9.1, 6.2, 3.7 Hz, 2H, dHh-5, 4″-H), 3.61-3.49 (m, 1H, Linker-OCH₂), 3.37 (d, J=33.8 Hz, 2H, Linker-OCH₂, 5″-H), 3.25 (d, J=27.4 Hz, 2H, Linker-NCH₂), 2.61-2.48 (m, 2H, RHb-2), 2.36 (dd, J=14.2, 5.7 Hz, 1H, dHh-2), 2.30-2.21 (m, 1H, dHh-2), 1.77-1.69 (m, 3H, NHAc—CH₃), 1.64-1.44 (m, 6H, dHh-4, Linker-CH₂), 1.34 (d, J=6.1 Hz, 3H, RHb-4), 1.33-1.21 (m, 2H, Linker-CH₂), 1.16 (dd, J=9.9, 6.1 Hz, 3H, 6-CH₃), 1.10 (d, J=6.1 Hz, 3H, 6″-CH₃), 1.06 (d, J=6.1 Hz, 3H, dHh-6), 0.95 (d, J=6.4 Hz, 3H, 6′-CH₃). ¹³C NMR (151 MHz, Methanol-d₄) δ 172.9 (NH—C═O), 172.0 (NH—C═O), 164.7 (NH—C═O), 141.8-137.4 (Ar), 136.3 (Ar), 133.1 (Ar), 126.0-123.8 (Ar), 100.7 (1″-C), 98.2 (1′-C), 97.0 (1-C), 78.5 (4-C, dHh-5), 76.0 (3-C, 3″-C), 74.9 (4′-C), 73.4 (dHh-3), 73.1 (RHb-3, Ar—CH₂), 72.1 (Ar—CH₂), 71.4 (4″-H), 70.9 (Ar—CH₂), 70.5 (5″-H), 69.9 (Ar—CH₂), 67.6 (Ar—CH₂), 67.1 (Ar—CH₂) 66.5 (5′-C, 5-C), 56.9 (2″-C), 56.2 (4″-C), 53.6 (4′-C), 50.1 (Ar-CH₂, 2-C), 46.4 (Linker-NCH₂), 43.1 (RHb-2), 42.9 (dHh-4), 41.4 (dHh-2), 28.8 (Linker-CH₂), 23.2 (Linker-CH₂), 21.8 (NHAc—CH₃), 18.8 (RHb-4, dHh-6), 17.1 (6″-C), 15.7 (6-CH₃, 6′-CH₃), HR-ESI-MS (m/z): calcd for C98H115Cl2N5O18Na⁺(M+Na)+: 1742.7506 found: 1742.7547.

Compound 34*: Under the protection of nitrogen, trisaccharide 31* (30 mg, 20.4 μmol) was dissolved in a mixed solution of water (1 mL), Et₃N (124.8 μl, 0.90 mmol) and pyridine (4 mL), and then, 1,3-propanedithiol (122 μl, 1.22 mmol) was added and stirred at room temperature for 6 h. Then, the reaction mixture was concentrated to obtain a crude product amino trisaccharide, and the next reaction was performed directly. 3,5-dibenzylhexanoic acid 20* (10 mg, 30.6 μmol) and amino trisaccharide were dissolved in an acetonitrile (5 mL) solution at room temperature, and sodium bicarbonate (5.1 mg, 61.2 μmol) was added. After 10 min, 1-hydroxybenzotriazole (0.55 mg, 4.08 μmol) and 1-ethyl-(3-(dimethylamino)propyl)carbodiimide (6.25 mg, 32.6 μmol) were added sequentially and stirred at room temperature for 6 h. After the reaction of raw materials was completed as detected by TLC, a crude product was dissolved in ethyl acetate and washed with a saturated sodium chloride solution, a separated organic layer was dried over anhydrous sodium sulfate, concentration was carried out under vacuum, and purification was performed by column chromatography (DCM:MeOH=20:1) to obtain compound 34* (21.4 mg, 12.4 μmol, 61%). [α]²⁵D=+8.5° (c=0.3, CHCl₃); IR νmax (film) 2956, 2857, 1739, 1660, 1543, 1499, 1464, 1374, 1240, 1081, 1046, 819, 757, 698 cm⁻¹; ¹H NMR (600 MHz, Methanol-d₄) δ 7.80-7.65 (m, 4H, Ar), 7.54-7.12 (m, 38H, Ar), 5.95 (s, 1H, DCA-H), 5.18-5.07 (m, 2H, Ar—CH₂), 4.97-4.84 (m, 4H, Ar—CH₂, 1′-H, 1-H, 1″-H), 4.77-4.64 (m, 4H, Ar—CH₂), 4.61-4.55 (m, 2H, Ar—CH₂), 4.53-4.36 (m, 7H, Ar—CH₂, 4′-H, 2-H), 4.30 (d, J=11.3 Hz, 1H, Ar—CH₂), 4.16 (dd, J=11.4, 6.2 Hz, 3H, Ar—CH₂, 3′-H, 5′-H), 4.05 (ddd, J=14.4, 7.1, 5.5 Hz, 2H, RHb-3, dHh-3), 4.00 (d, J=23.2 Hz, 1H, 3-H), 3.93-3.84 (m, 2H, 4-H, 5-H), 3.82-3.71 (m, 3H, 2″-H, 4″-H, 2′-H), 3.70-3.50 (m, 3H, 3″-H, dHh-5, Linker-OCH₂), 3.36 (s, 2H, Linker-OCH₂, 5″-H), 3.23 (s, 2H, Linker-NCH₂), 2.62-2.46 (m, 2H, RHb-2), 2.37-2.22 (m, 2H, dHh-2), 1.72 (d, J=7.8 Hz, 3H, NHAc—CH₃), 1.64-1.46 (m, 6H, dHh-4, Linker-CH₂), 1.35 (d, J=6.2 Hz, 3H, RHb-4), 1.28 (s, 2H, Linker-CH₂), 1.16 (t, J=7.9 Hz, 3H, 6-CH₃), 1.13-1.02 (m, 3H, 6″-CH₃), 0.97 (dd, J=25.3, 6.3 Hz, 6H, dHh-CH₃, 6′-CH₃). ¹³C NMR (151 MHz, Methanol-d₄) δ 172.9 (NH—C═O), 172.2 (NH—C═O), 171.9 (NH—C═O), 164.7 (NH—C═O), 138.8 (Ar), 138.5 (Ar), 138.2 (Ar), 135.8 (Ar), 133.3 (Ar), 133.0 (Ar), 128.3 (Ar), 128.2 (Ar), 127.9 (Ar), 127.3 (Ar), 127.1 (Ar), 125.8 (Ar), 125.6 (Ar), 125.4 (Ar), 100.6 (1″-C), 98.2 (1′-C), 97.1 (1-C), 78.5 (3″-C), 76.2 (3-C, 2′-C), 74.9 (3′-C), 73.6 (Ar—CH₂), 72.9 (RHb-3, dHh-3), 71.4 (Ar—CH₂), 71.3 (dHh-5, Ar—CH₂), 70.5 (5″-C, Ar—CH₂), 69.8 (Ar—CH₂), 67.6 (Ar—CH₂), 66.6 (5-C, Ar—CH₂), 66.4 (5′-C) 57.07 (2″-C), 55.9 (4″-C), 53.6 (4′-C), 50.1 (Ar-CH₂, 2-C), 46.6 (Linker-NCH₂), 43.1 (RHb-2), 42.4 (dHh-4), 41.8 (dHh-2), 28.7 (Linker-CH₂), 23.1 (Linker-CH₂), 21.8 (NHAc—CH₃), 18.8 (RHb-4), 18.7 (dHh-6), 17.0 (6″-C), 15.8 (6-C), 15.7 (6″-C). HR-ESI-MS (m/z): calcd for C98H115Cl2N5O18Na⁺(M+Na)+: 1742.7506 found: 1742.7512.

Compound 35*: Under the protection of nitrogen, trisaccharide 31* (30 mg, 20.4 μmol) was dissolved in a mixed solution of water (1 mL), Et₃N (124.8 μl, 0.90 mmol) and pyridine (4 mL), and then, 1,3-propanedithiol (122 μl, 1.22 mmol) was added and stirred at room temperature for 6 h. Then, the reaction mixture was concentrated to obtain a crude product amino trisaccharide, and the next reaction was performed directly. 3,5-dibenzylhexanoic acid 21* (10 mg, 30.6 μmol) and amino trisaccharide were dissolved in an acetonitrile (5 mL) solution at room temperature, and sodium bicarbonate (5.1 mg, 61.2 μmol) was added. After 10 min, 1-hydroxybenzotriazole (0.55 mg, 4.08 μmol) and 1-ethyl-(3-(dimethylamino)propyl)carbodiimide (6.25 mg, 32.6 μmol) were added sequentially and stirred at room temperature for 6 h. After the reaction of raw materials was completed as detected by TLC, a crude product was dissolved in ethyl acetate and washed with a saturated sodium chloride solution, a separated organic layer was dried over anhydrous sodium sulfate, concentration was carried out under vacuum, and purification was performed by column chromatography (DCM:MeOH=20:1) to obtain compound 35* (23.86 mg, 13.87 μmol, 68%). [α]²⁵D=−7.8° (c=0.3, CHCl₃); IR νmax (film) 2989, 2851, 1749, 1660, 1545, 1499, 1454, 1384, 1240, 1081, 1046, 819, 757, 698 cm⁻¹; ¹H NMR (600 MHz, Methanol-d₄) δ 7.77-7.66 (m, 4H, Ar), 7.45-7.13 (m, 39H, Ar), 5.95 (s, 1H, DCA-CH), 5.17-5.07 (m, 2H, Ar—CH₂), 4.98-4.84 (m, 4H, 1′-H, 1-H, 1″-H, Ar—CH₂), 4.75 (d, J=11.1 Hz, 1H, Ar—CH₂), 4.71-4.64 (m, 3H, Ar—CH₂), 4.59 (d, J=11.3 Hz, 2H, Ar—CH₂), 4.49 (d, J=12.0 Hz, 3H, Ar—CH₂), 4.45-4.35 (m, 5H, Ar—CH₂, 4′-H, 2-H), 4.29 (d, J=11.5 Hz, 1H, Ar—CH₂), 4.19-4.10 (m, 2H, 3′-H, 5′-H), 4.06 (h, J=6.4 Hz, 1H, RHb-3), 3.98 (p, J=6.3 Hz, 2H, dHh-3, 3-H), 3.88 (dt, J=21.2, 8.7 Hz, 2H, 4-H, 5-H), 3.77 (dd, J=10.3, 3.9 Hz, 3H, 2″-H, 2′-H, 4″-H), 3.62 (dd, J=28.1, 7.7 Hz, 2H, 3″-H, Linker-OCH₂), 3.49 (p, J=6.2 Hz, 1H, dHh-5), 3.35 (m, 2H, 5″-H, Linker-OCH₂), 3.23 (m, 2H, Linker-NCH₂), 2.55 (ddd, J=47.7, 14.3, 6.3 Hz, 2H, RHb-2), 2.26 (pd, J=15.3, 14.4, 8.6 Hz, 2H, dHh-2), 1.88 (dq, J=13.4, 6.2 Hz, 1H, dHh-4), 1.78-1.66 (m, 3H, NHAc—CH₃), 1.65-1.42 (m, 5H, Linker-CH₂, dHh-4), 1.35 (d, J=6.1 Hz, 3H, RHb-4), 1.28 (m, 2H, Linker-CH₂), 1.17 (d, J=6.1 Hz, 3H, 6-CH₃), 1.04 (dd, J=13.5, 6.1 Hz, 6H, 6″-CH₃, dHh-6), 0.95 (d, J=6.4 Hz, 3H, 6′-CH₃). ¹³C NMR (151 MHz, Methanol-d₄) δ 172.9 (NH—C═O), 172.3 (NH—C═O), 164.8 (NH—C═O), 139.6-137.7 (Ar), 135.9 (Ar), 133.1 (Ar), 126.2-125.1 (Ar), 100.6 (1″-H), 98.3 (1′-H), 97.1 (1-H), 78.6 (3″-C), 76.2 (3-C), 74.9 (3′-C, 2′-C), 73.7 (dHh-3), 72.9 (RHb-3), 71.7 (dHh-5), 70.8 (5″-H, Ar—CH₂), 70.5 (Ar—CH₂), 69.9 (Ar—CH₂), 67.6 (Ar—CH₂), 66.8 (5-C) 66.5 (5′-C), 57.1 (2″-C), 55.8 (4″-C), 53.6 (4′-C), 50.1 (Ar-CH₂, 2-C), 46.2 (Linker-NCH₂), 43.1 (RHb-2), 41.4 (dHh-2), 40.7 (dHh-4), 28.8 (Linker-CH₂), 23.0 (Linker-CH₂) 21.8 (NHAc—CH₃), 18.8 (RHb-4), 18.6 (dHh-6), 17.0 (6″-C), 15.7 (6-C), 15.7 (6′-C). HR-ESI-MS (m/z): calcd for C98H115Cl2N5O18Na⁺(M+Na)+: 1742.7506 found: 1742.7526.

### Example 5

### Synthesis of Compounds 1*, 2*, 3*, 4*, and 5*

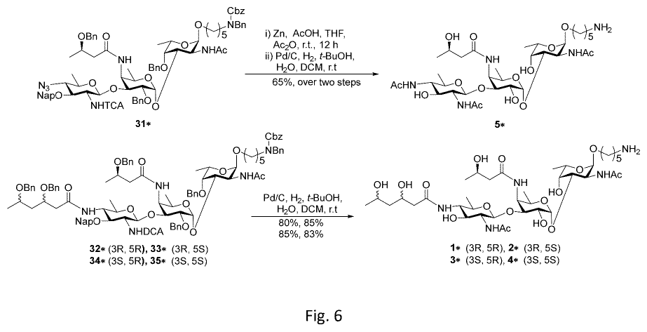

As shown in FIG. 6: Fully protected trisaccharides 32*, 33*, 34*, and 35* were subjected to deprotection under the condition of palladium-on-carbon hydrogenation to synthesize four different configurations of target trisaccharides 1*, 2*, 3*, and 4*. The azide group of trisaccharide 31* was converted to NHAc in the presence of zinc powder, acetic acid, and acetic anhydride, and then, deprotection was performed directly by palladium-on-carbon hydrogenation to obtain trisaccharide 5*.

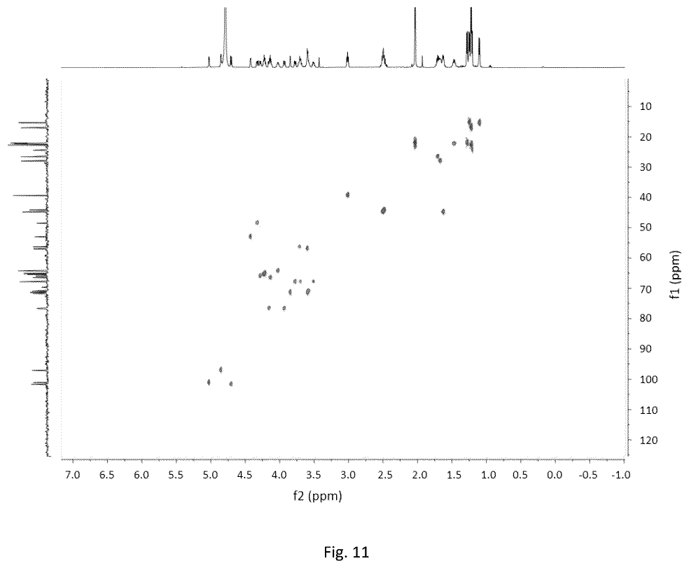

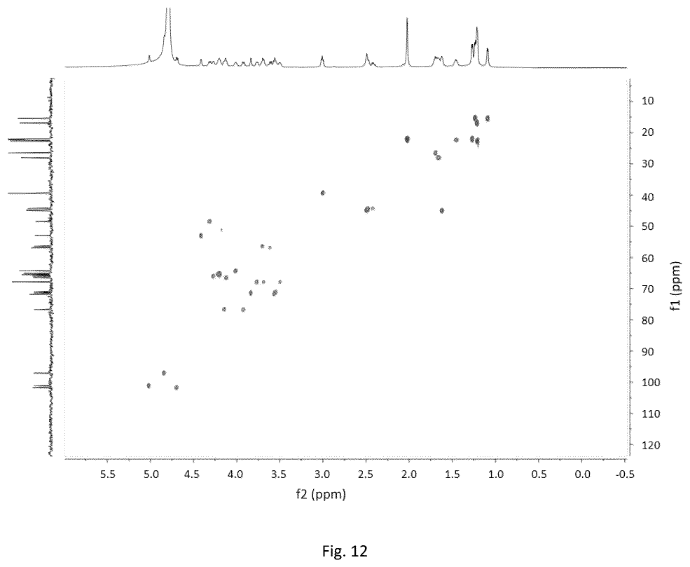

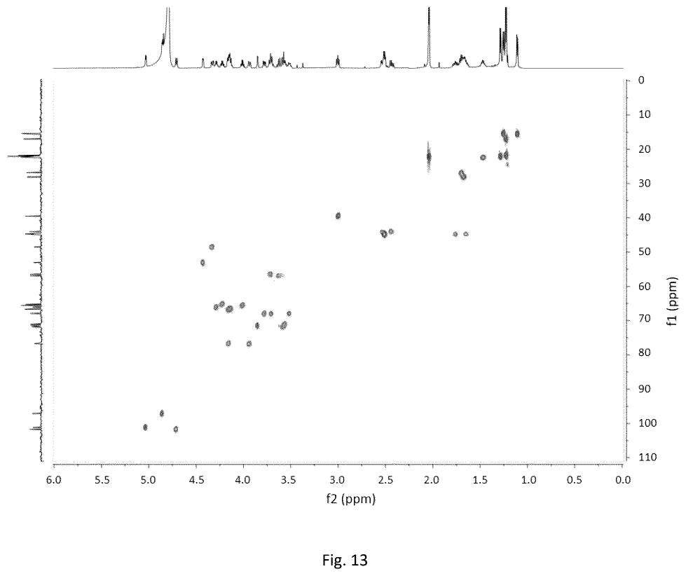

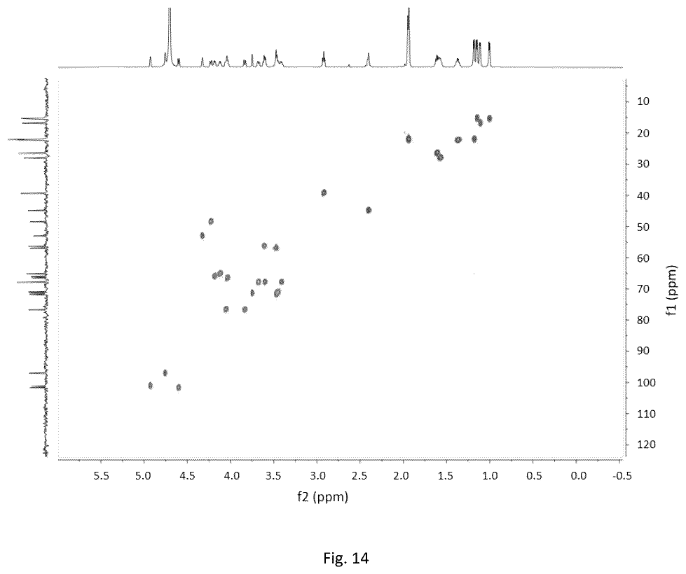

NMR two-dimensional HSQC spectra of compounds 1*, 2*, 3*, 4*, and 5* were shown in FIG. 10 to FIG. 14.

### Specific Experiment Operations and Steps:

Compound 1*: Trisaccharide 32* (12 mg, 6.0 μmol) was dissolved in a mixed solution of dichloromethane, tert-butyl alcohol, and water (3:6:1, v/v/v, 3 mL). Air in a reaction flask was replaced with nitrogen, an appropriate amount of 10% palladium-on-carbon was added, and the solution was purged with hydrogen for 5 min, then stirred under the hydrogen atmosphere for 24 h, filtered with diatomite and concentrated. The residue was purified by HPLC using a semi-preparative (Thermo Scientific Hypercarb) column at a flow rate of 1 mL/min, and was eluted with ultrapure water containing 0.1% formic acid (solvent A) and acetonitrile (solvent B) in a linear gradient of 10% to 30% solvent B for 30 min to obtain compound 1* (4.67 mg, 5.58 μmol, 80%). ¹H NMR (600 MHz, Deuterium Oxide) δ 5.00 (d, J=4.3 Hz, 1H, 1′-H), 4.83 (d, 1H, 1-H), 4.68 (d, J=8.4 Hz, 1H, 1″-H), 4.40 (d, J=4.8 Hz, 1H, 4′-H), 4.33-4.23 (m, 2H, 2-H, 5′-H), 4.23-4.08 (m, 4H, RHb-3, dHh-3, 3′-H, 5-H), 3.98 (h, J=6.5 Hz, 1H, dHh-5), 3.91 (dd, J=11.0, 3.1 Hz, 1H, 3-H), 3.82 (d, J=3.2 Hz, 1H, 4-H), 3.75 (dd, J=10.6, 4.3 Hz, 1H, 2′-H), 3.68 (td, J=10.0, 5.5 Hz, 2H, 2″-H, Linker-OCH₂), 3.57 (q, J=7.3, 6.2 Hz, 3H, 3″-H, 4″-H, 5″-H), 3.48 (dt, J=10.1, 6.4 Hz, 1H, Linker-OCH₂), 3.02-2.96 (m, 2H, Linker-NCH₂), 2.54-2.41 (m, 4H, RHb-2, dHh-2), 2.01 (d, J=4.1 Hz, 6H, NHAc—CH₃), 1.78-1.55 (m, 6H, dHh-4, Linker-CH₂), 1.45 (dq, J=14.9, 7.4, 7.0 Hz, 2H, Linker-CH₂), 1.26 (d, J=6.3 Hz, 3H, RHb-4), 1.24-1.15 (m, 9H, 6-CH₃, 6″-CH₃, dHh-6), 1.08 (d, J=6.5 Hz, 3H, 6′-CH₃). ¹³C NMR (151 MHz, Deuterium Oxide) δ 174.7 (NH—C═O), 174.2 (NH—C═O), 171.0 (NH—C═O), 101.7 (1″-H), 101.1 (1′-H), 97.1 (1-H), 76.7 (3-C), 76.6 (3′-C), 71.7 (5″-C), 71.4 (4-C), 70.9 (3″-C), 67.8 (2′-C, Linker-CH₂), 66.6 (dHh-3), 66.5 (5-C), 66.0 (5′-C), 65.5 (dHh-5), 65.2 (RHb-3), 57.0 (4″-C), 56.4 (2″-C), 53.0 (4′-C), 48.5 (2-C), 44.8 (RHb-2), 44.5 (dHh-4), 43.8 (dHh-2), 39.4 (Linker-NCH₂), 28.0 (Linker-CH₂), 26.5 (Linker-CH₂), 22.3 (NHAc—CH₃), 22.2 (Linker-CH₂), 22.0 (RHb-4), 21.7 (6″-CH₃), 17.0 (dHh-6), 15.5 (6′-CH₃), 15.3 (6-CH₃). HR-ESI-MS (m/z): calcd for C37H67N5O16Na⁺(M+Na)+: 860.4475 found: 860.4478

Compound 2*: Trisaccharide 33* (17 mg, 9.89 μmol) was dissolved in a mixed solution of dichloromethane, tert-butyl alcohol, and water (3:6:1, v/v/v, 3 mL). Air in a reaction flask was replaced with nitrogen, an appropriate amount of 10% palladium-on-carbon was added, and the solution was purged with hydrogen for 5 min, then stirred under the hydrogen atmosphere for 24 h, filtered with diatomite and concentrated. The residue was purified by HPLC using a semi-preparative (Thermo Scientific Hypercarb) column at a flow rate of 1 mL/min, and was eluted with ultrapure water containing 0.1% formic acid (solvent A) and acetonitrile (solvent B) in a linear gradient of 10% to 30% solvent B for 30 min to obtain compound 2* (7 mg, 8.40 μmol, 85%). ¹H NMR (600 MHz, Deuterium Oxide) δ 5.03 (d, J=4.3 Hz, 1H, 1′-H), 4.85 (d, J=3.8 Hz, 1H, 1-H), 4.70 (d, J=8.4 Hz, 1H, 1″-H), 4.44-4.40 (m, 1H, 4′-H), 4.32 (dd, J=11.1, 3.8 Hz, 1H, 2-H), 4.30-4.26 (m, 1H, 5′-H), 4.22 (p, J=6.8 Hz, 2H, RHb-3, dHh-3), 4.14 (m, J=13.5, 12.0, 5.6 Hz, 2H, 3′-H, 5-H), 4.02 (q, J=6.3 Hz, 1H, dHh-5), 3.93 (dd, J=11.0, 3.1 Hz, 1H, 3-H), 3.85 (d, J=3.2 Hz, 1H, 4-H), 3.77 (dd, J=10.6, 4.2 Hz, 1H, 2′-H), 3.70 (m, J=8.1 Hz, 2H, 2″-H, Linker-OCH₂), 3.59 (d, J=6.0 Hz, 3H, 3″-H, 4″-H, 5″-H), 3.51 (m, J=10.3, 6.2 Hz, 1H, Linker-OCH₂), 3.02 (t, J=7.7 Hz, 2H, Linker-NCH₂), 2.54-2.43 (m, 4H, RHb-2, dHh-2), 2.03 (d, J=4.4 Hz, 6H, NHAc—CH₃), 1.70 (td, J=15.4, 7.7 Hz, 4H, Linker-CH₂), 1.63 (dt, J=7.9, 3.9 Hz, 2H, dHh-4), 1.47 (q, J=7.1 Hz, 2H, Linker-CH₂), 1.28 (d, J=6.3 Hz, 3H, RHb-4), 1.25 (d, J=6.5 Hz, 3H, 6-CH₃), 1.22 (d, J=6.0 Hz, 6H, dHh-6, 6″-CH₃), 1.10 (d, J=6.5 Hz, 3H, 6′-CH₃). ¹³C NMR (151 MHz, Deuterium Oxide) δ 174.7 (NH—C═O), 174.6 (NH—C═O), 174.5 (NH—C═O), 174.2 (NH—C═O), 101.7 (1″-C), 101.1 (1′-C), 97.1 (1-C), 76.7 (3-C), 76.6 (3′-C), 71.7 (5″-C), 71.4 (4-C), 70.9 (3″-C), 67.8 (2′-C, Linker-OCH₂), 66.5 (5-C), 66.0 (5′-C), 65.5 (RHb-3), 65.2 (dHh-3), 64.3 (dHh-5), 57.0 (4″-C), 56.4 (2″-C), 53.0 (4′-C), 48.5 (2-C), 44.8-44.8 (RHb-2, dHh-4), 44.1 (dHh-2), 39.4 (Linker-NCH₂), 28.0 (Linker-CH₂), 26.5 (Linker-CH₂), 22.7 (dHh-6-CH₃), 22.3 (Linker-CH₂), 22.2 (NHAc—CH₃), 22.0 (NHAc—CH₃), 22.02 (RHb-4-CH₃), 17.0 (6″-CH₃), 15.5 (6′-CH₃), 15.3 (6-CH₃). HR-ESI-MS (m/z): calcd for C37H67N5O16Na⁺(M+Na)+: 860.4475 found: 860.4490

Compound 3*: Trisaccharide 34* (9 mg, 3.1 μmol) was dissolved in a mixed solution of dichloromethane, tert-butyl alcohol, and water (3:6:1, v/v/v, 3 mL). Air in a reaction flask was replaced with nitrogen, an appropriate amount of 10% palladium-on-carbon was added, and the solution was purged with hydrogen for 5 min, then stirred under the hydrogen atmosphere for 24 h, filtered with diatomite and concentrated. The residue was purified by HPLC using a semi-preparative (Thermo Scientific Hypercarb) column at a flow rate of 1 mL/min, and was eluted with ultrapure water containing 0.1% formic acid (solvent A) and acetonitrile (solvent B) in a linear gradient of 10% to 30% solvent B for 30 min to obtain compound 3* (2.2 mg, 2.64 μmol, 85%). ¹H NMR (600 MHz, Deuterium Oxide) δ 5.01 (d, J=4.2 Hz, 1H, 1′-H), 4.84 (d, 1H, 1-H), 4.69 (d, J=8.6 Hz, 1H, 1″-H), 4.41 (m, 1H, 4′-H), 4.31 (dd, J=11.1, 3.6 Hz, 1H, 2-H), 4.27 (t, J=6.8 Hz, 1H, 5′-H), 4.20 (m, J=6.6 Hz, 2H, RHb-3, dHh-3), 4.13 (dq, J=14.2, 7.1, 5.8 Hz, 2H, 3′-H, 5-H), 4.01 (q, J=6.4 Hz, 1H, dHh-5), 3.92 (d, J=11.3 Hz, 1H, 3-H), 3.84 (m, 1H, 4-H), 3.76 (dd, J=10.7, 4.1 Hz, 1H, 2′-H), 3.70 (t, J=9.5 Hz, 2H, 2″-H, Linker-OCH₂), 3.62 (t, J=9.9 Hz, 1H, 4″-H), 3.56 (t, J=9.8 Hz, 2H, 3″-H, 5″-H), 3.50 (m, 1H, Linker-OCH₂), 3.01 (t, J=7.9 Hz, 2H, Linker-NCH₂), 2.53-2.38 (m, 4H, RHb-2, dHh-2), 2.02 (d, J=3.3 Hz, 6H, NHAc), 1.74-1.59 (m, 6H, Linker-CH₂, dHh-4), 1.46 (dt, J=15.0, 7.5 Hz, 2H, Linker-CH₂), 1.27 (m, J=6.3 Hz, 3H, RHb-4), 1.22 (m, J=15.0, 6.3 Hz, 9H, 6-CH₃, 6″-CH₃, dHh-6), 1.09 (d, J=6.5 Hz, 3H, 6′-CH₃). ¹³C NMR (151 MHz, Deuterium Oxide) δ 174.6 (NH—C═O), 174.6 (NH—C═O), 174.4 (NH—C═O), 101.7 (1″-H), 101.1 (1′-H), 97.1 (1-H), 76.7 (3-C), 76.6 (3′-C), 71.8 (5″-C), 71.4 (4-C), 70.1 (3″-C), 67.82 (2′-C, Linker-OCH₂), 66.5 (5-C), 66.0 (5′-C), 65.6-65.2 (RHb-3, dHh-3), 64.3 (dHh-5), 56.9 (4″-C), 56.4 (2″-C), 53.0 (4′-C), 48.5 (2-C), 45.0 (dHh-4), 44.8 (RHb-2), 44.3 (dHh-2), 39.3 (Linker-NCH₂), 28.0 (2-C), 26.5 (2-C), 22.7 (dHh-6), 22.3 (Linker-CH₂), 22.2 (NHAc—CH₃), 22.0 (RHb-4), 17.0 (6″-CH₃), 15.5 (6′-CH₃), 15.3 (6-CH₃). HR-ESI-MS (m/z): calcd for C37H67N5O16Na⁺(M+Na)+: 860.4475 found: 860.4476

Compound 4*: Trisaccharide 35* (7.8 mg, 4.536 μmol) was dissolved in a mixed solution of dichloromethane, tert-butyl alcohol, and water (3:6:1, v/v/v, 3 mL). Air in a reaction flask was replaced with nitrogen, an appropriate amount of 10% palladium-on-carbon was added, and the solution was purged with hydrogen for 5 min, then stirred under the hydrogen atmosphere for 24 h, filtered with diatomite and concentrated. The residue was purified by HPLC using a semi-preparative (Thermo Scientific Hypercarb) column at a flow rate of 1 mL/min, and was eluted with ultrapure water containing 0.1% formic acid (solvent A) and acetonitrile (solvent B) in a linear gradient of 10% to 30% solvent B for 30 min to obtain compound 4* (3.15 mg, 3.765 μmol, 83%). ¹H NMR (600 MHz, Deuterium Oxide) δ 5.03 (d, J=4.3 Hz, 1H, 1′-H), 4.86 (s, 1H, 1-H), 4.71 (d, J=8.4 Hz, 1H, 1″-H), 4.43 (d, J=4.7 Hz, 1H, 4′-H), 4.33 (dd, J=11.2, 3.8 Hz, 1H, 2-H), 4.29 (d, J=6.6 Hz, 1H, 5′-H), 4.22 (q, J=6.4 Hz, 1H, RHb-3), 4.18-4.11 (m, 3H, dHh-3, 3′-H, 5-H), 4.01 (q, J=6.4 Hz, 1H, dHh-5), 3.94 (dd, J=11.2, 3.2 Hz, 1H, 3-H), 3.85 (d, J=3.2 Hz, 1H, 4-H), 3.78 (dd, J=10.6, 4.2 Hz, 1H, 2′-H), 3.71 (q, J=8.7, 8.0 Hz, 2H, 2″-H, Linker-OCH₂), 3.65-3.61 (m, 1H, 4″-H), 3.60-3.54 (m, 2H, 3″-H, 5″-H), 3.52 (m, J=10.4, 6.4, 5.2 Hz, 1H, Linker-OCH₂), 3.07-2.97 (m, 2H, Linker-NCH₂), 2.55-2.49 (m, 3H, RHb-2, dHh-2), 2.43 (dd, J=14.2, 8.5 Hz, 1H, dHh-2), 2.04 (d, J=3.7 Hz, 6H, NHAc—CH₃), 1.78-1.63 (m, 6H, dHh-4, Linker-CH₂), 1.47 (m, J=7.2 Hz, 2H, Linker-CH₂), 1.29 (d, J=6.3 Hz, 3H, RHb-4), 1.25 (d, J=6.6 Hz, 3H, 6-CH₃), 1.23 (d, J=6.1 Hz, 6H, 6″-CH₃, dHh-6), 1.11 (d, J=6.5 Hz, 3H, 6′-CH₃). ¹³C NMR (151 MHz, Deuterium Oxide) δ 174.7 (NH—C═O), 174.6 (NH—C═O), 174.6 (NH—C═O), 174.3 (NH—C═O), 101.7 (1″-C), 101.2 (1′-C), 97.1 (1-C), 76.7 (3-C), 76.6 (3′-C), 71.8 (5″-C), 71.4 (4-C), 71.0 (3″-C), 67.9 (2′-C, Linker-OCH₂), 66.7 (dHh-3), 66.5 (C-5), 66.1 (5′-C), 65.5 (dHh-5), 65.2 (RHb-3), 56.9 (4″-C), 56.5 (2″-C), 53.0 (4′-C), 48.5 (2-C), 44.9 (dHh-4), 44.7 (RHb-2), 44.0 (dHh-2), 39.5 (Linker-NCH₂), 28.1 (Linker-CH₂), 26.8 (Linker-CH₂), 22.3 (Linker-CH₂), 22.2 (NHAc—CH₃), 22.1 (RHb-4), 21.8 (dHh-6), 17.0 (6″-CH₃), 15.5 (6′-CH₃), 15.3 (6-CH₃). HR-ESI-MS (m/z): calcd for C37H67N5O16Na⁺(M+H)+: 838.4656 found: 838.4647

Compound 5*: Trisaccharide 31* (30 mg, 20.41 μmol) was dissolved in a mixed solution of tetrahydrofuran, acetic anhydride and acetic acid (3/2/1, v/v/v, 3 mL), newly activated Zn (1 g) was added, and the solution was stirred at room temperature overnight. After the reaction of raw materials was completed as detected by TLC, the reaction solution was diluted with dichloromethane and filtered. The filtrate was washed with a saturated sodium bicarbonate solution and a saturated sodium chloride solution. Then, the combined organic layers were dried over anhydrous sodium sulfate, filtered, evaporated in vacuum, and dried by evacuating on an oil pump. A crude product was dissolved in a mixed solution of dichloromethane, tert-butyl alcohol and water (3/6/1, v/v/v, 2 mL), and an appropriate amount of 10% palladium-on-carbon was added to the solution. The solution was stirred for 36 h under the hydrogen atmosphere (4 atm), then the mixture was filtered with diatomite and washed with water three times, and the solvent was evaporated in vacuum. The residue was purified by HPLC using a semi-preparative (Thermo Scientific Hypercarb) column at a flow rate of 1 mL/min, and was eluted with ultrapure water containing 0.1% formic acid (solvent A) and acetonitrile (solvent B) in a linear gradient of 10% to 30% solvent B for 30 min to obtain compound 5* (9.9 mg, 13.27 μmol, 65% yield over two steps). ¹H NMR (600 MHz, Deuterium Oxide) δ 4.93 (t, J=3.3 Hz, 1H, 1′-H), 4.76 (d, J=3.3 Hz, 1H, 1-H), 4.60 (dd, J=7.8, 2.0 Hz, 1H, 1″-H), 4.33 (d, J=4.7 Hz, 1H, 4′-H), 4.23 (d, J=11.3, 3.1 Hz, 1H, 2-H), 4.18 (m, J=6.8 Hz, 1H, 5-H), 4.12 (q, J=6.1 Hz, 1H, RHb-3), 4.08-4.00 (m, 2H, 3′-H, 5′-H), 3.83 (dt, J=11.1, 2.9 Hz, 1H, 3-H), 3.75 (d, J=3.0 Hz, 1H, 4-H), 3.68 (dt, J=10.6, 3.2 Hz, 1H, 2′-H), 3.60 (m, J=7.9, 7.4 Hz, 2H, 2″-H, Linker-OCH₂), 3.44 (m, J=28.2, 9.7, 4.4 Hz, 4H, 3″-H, 4″-H, 5″-H, Linker-OCH₂), 2.98-2.89 (m, 2H, Linker-NCH₂), 2.45-2.36 (m, 2H, RHb-2), 1.94 (dt, J=6.4, 2.2 Hz, 9H, NHAc—CH₃), 1.66-1.54 (m, 4H, Linker-CH₂), 1.37 (q, J=7.4 Hz, 2H, Linker-CH₂), 1.18 (m, 3H, RHb-4), 1.15 (m, 3H, 6′-CH₃), 1.11 (m, 3H, 6″-CH₃) 1.00 (m, 3H, 6-CH₃). ¹³C NMR (151 MHz, Deuterium Oxide) δ 174.6 (NH—C═O), 171.0 (NH—C═O), 101.7 (1″-H), 101.1 (1′-H), 97.1 (1′-H), 76.7 (3′-C, 3-C), 71.8 (4-C), 71.4 (3″-C), 70.9 (5″-C), 67.8 (2′-H, Linker-OCH₂), 66.5 (5′-C), 66.0 (5-C), 65.2 (RHb-3) 57.0 (4″-C), 56.3 (2″-H), 44.8 (RHb-2), 39.3 (Linker-NCH₂), 28.0 (Linker-CH₂), 26.5 (Linker-CH₂), 22.3 (Linker-CH₂), 22.2 (NHAc—CH₃), 22.1 (NHAc—CH₃), 22.0 (NHAc—CH₃), 16.9 (6″-H), 15.5 (6-H), 15.3 (6′-CH₃). HR-ESI-MS (m/z): calcd for C33H59N5O14Na⁺(M+Na)+: 772.3951 found: 772.3968.

### Example 6

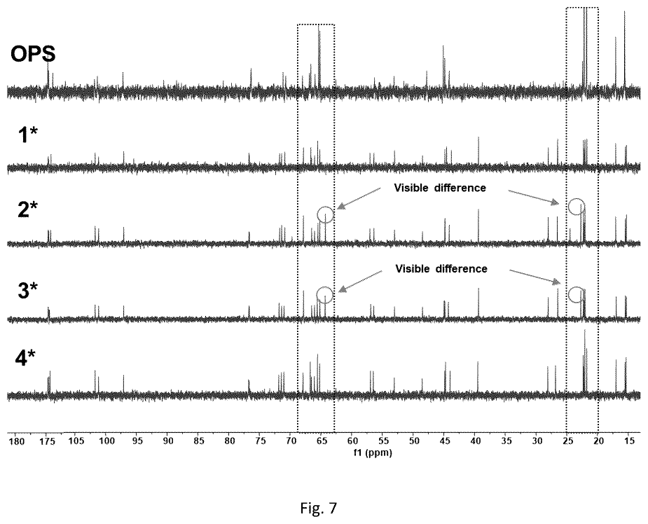

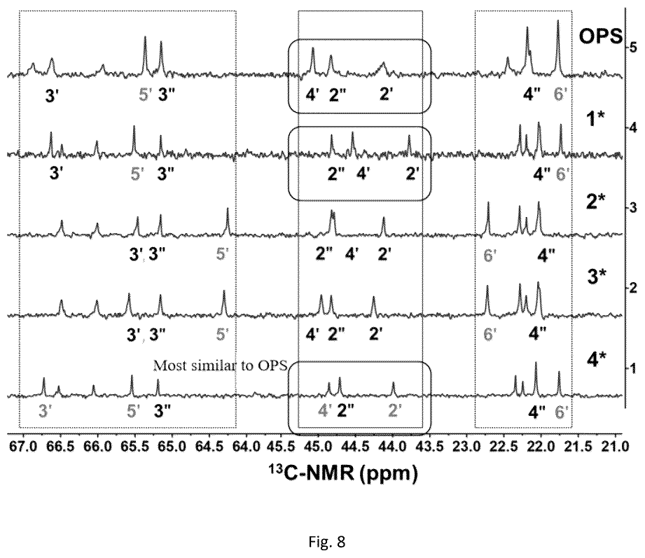

The absolute configuration of dHh was illustrated through NMR analysis by using four synthetic oligosaccharides, as shown in FIG. 7 to FIG. 8.

Specific analysis processes: In order to reduce the error as much as possible, nuclear magnetic testing instruments used for synthetic oligosaccharides (1* to 4*) were all 600 M, and tests were performed at the same temperature (25° C.). The NMR spectra of the four synthetic oligosaccharides and natural OPS were compared to attempt to determine the potential absolute configuration of a dHh side chain. It was worth noting that none of the four synthetic trisaccharides (1* to 4*) showed the same NMR-¹H and NMR-¹³C data as the naturally extracted O-antigen (OPS) trisaccharide repeating unit. It was speculated that this phenomenon was mainly attributed to the differences in length of the synthetic oligosaccharides and natural OPS, and the presence of an amino linker at the reducing end and the hydroxyl group at the 3 position of the non-reducing end D-quinovose (D-Qui-C3-OH) also increases the error. The RHb side chain, situated in the central region of the trisaccharide backbone, was employed as a control due to the fact that it is considered less susceptible to the effects of Linker and D-Qui-C3-OH. NMR-¹³C analysis showed that RHb of the four synthetic trisaccharides was more correlated with natural trisaccharides, while the NMR-¹³C chemical shift of dHh was significantly different from natural trisaccharides. Compared with synthetic sugars 2* and 3*, the difference in dHh-NMR-¹³C chemical shift between synthetic sugars 1* and 4* and natural trisaccharides was relatively small. As shown in FIG. 7, preliminary analysis can determine that synthetic sugars 1 and 4* were the most potential natural configurations. Through the integration and processing of NMR spectra, further analysis revealed that as shown in FIG. 8, four isomers exhibited visible differences of NMR-¹³C in dHh-2 and 4, while synthetic sugar 4* showed a better matching degree with the natural configuration. Therefore, the absolute configurations of dHh modifying groups in V. cholerae serotype O100 OPS were assigned as 3S and 5S.

### Example 7

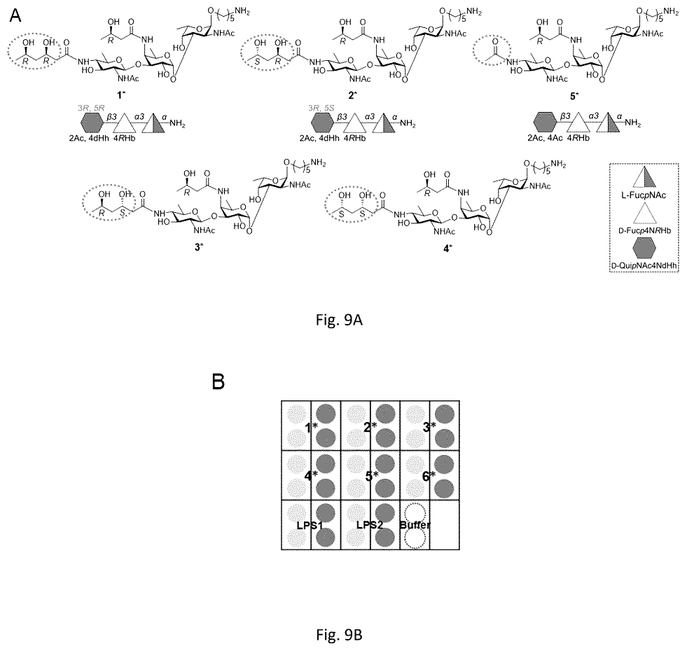

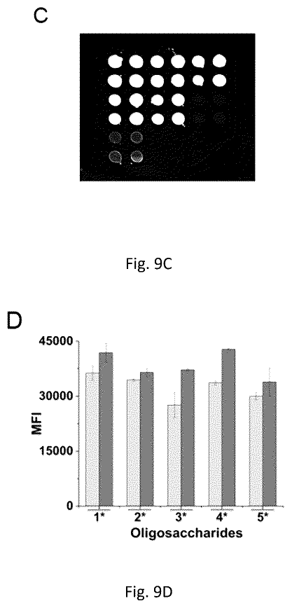

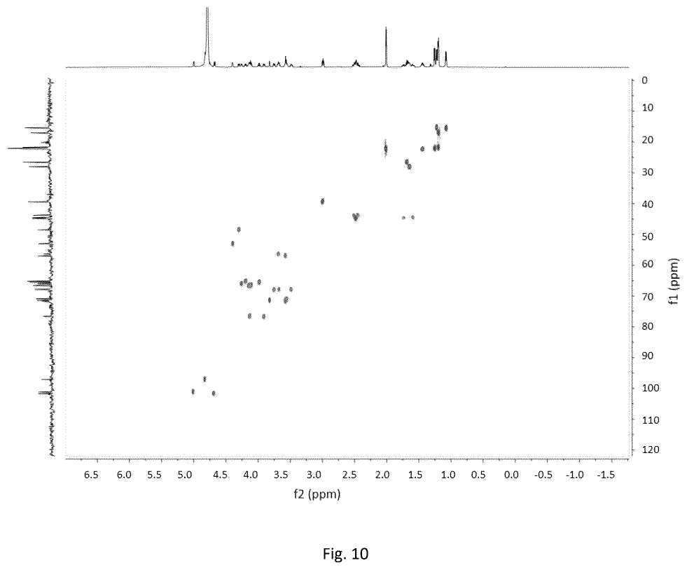

Immunological evaluation was performed by five synthetic oligosaccharides, as shown in FIG. 9A-D.

Specific steps and methods were as follows:

Preparation of glycan microarray: Five synthetic oligosaccharides (1* to 5*, FIG. 9A) and uncorrelated synthetic sugar (6*, D-FucNAc) were dissolved in a 50 mM phosphate solution (pH 8.5) to be prepared into 0.1 mM and 0.5 mM oligosaccharide solutions respectively, and 1 mg/mL and 5 mg/ml V. cholerae serotype O100 lipopolysaccharide (LPS1) and Proteus mirabilis lipopolysaccharide (LPS2) solutions were prepared correspondingly. Then, a microarray spotter was used for printing distribution grids shown in FIG. 9B onto the area of a microarray (9 mm long*9 mm wide), and incubation was performed overnight at room temperature and 65% humidity to covalently bind oligosaccharide fragments to the microarray. After incubation, a mixed solution (pH=9) of 100 nM ethanolamine and 50 nM sodium phosphate was used for processing at 50° C. for 1 h, and then, ultrapure water was used for washing.

The glycan microarray was blocked with a PBS solution containing 3% BSA at room temperature for 1 h. The glycan microarray was washed once with a PBS solution containing 0.1% Tween 20 (PBST solution), washed twice with a PBS solution, and spin-dried, and the glycan microarray was loaded into a 16-well incubator (ProPlate). 120 microliters of rabbit serum sample diluted in a ratio of 1:200 in a PBS solution containing 1% BSA was added to each well, and incubated overnight in a moist chamber at 4° C. in the dark to allow rabbit serum IgG antibodies identifying the synthetic oligosaccharide fragments to bind to the synthetic oligosaccharides on the glycan microarray, and the sample was removed and washed three times with 200 microliters of PBST solution to remove unbound serum IgG antibodies. Then, goat anti-rabbit serum IgG antibodies fluorescently labeled with cy3 were used as secondary antibodies, 120 microliters of secondary antibodies diluted in a ratio of 1:400 in a PBS solution containing 1% BSA were added to each well, and incubated in a moist chamber at room temperature in the dark for 45 min, and the secondary antibody solution was removed. 200 microliters of PBST solution was used for washing three times to remove unbound secondary antibodies, and the 16-well incubator was disassembled, washed once with ultrapure water, and then washed 15 min with ultrapure water to obtain a final detection microarray.

Scanning was performed on a microarray scanner to emit fluorescence signals, and results (FIG. 9C and FIG. 9D) showed that IgG antibodies exhibited excellent binding ability to all synthetic sugars 1* to 5*. Synthetic sugar 6* and LPS2 did not bind to antibodies, as expected. Compared with trisaccharide 4*, synthetic sugars 1* to 3* and 5* did not show a loss or significant decrease in binding ability.

Although the disclosure has been disclosed above with preferred examples, it is not intended to limit the disclosure. Anyone familiar with this technology can make various changes and modifications without departing from the principle and scope of the disclosure, therefore, the protection scope of the disclosure should be defined by the claims.

What is claimed is:

1. A chemical synthesis method of V. cholerae serotype O100 O-antigen oligosaccharides, comprising using three monosaccharide building blocks and five carboxylic acid derivatives as raw materials;
wherein the structures of the V. cholerae serotype O100 O-antigen oligosaccharides are represented by the following Formulae (1) to (5): 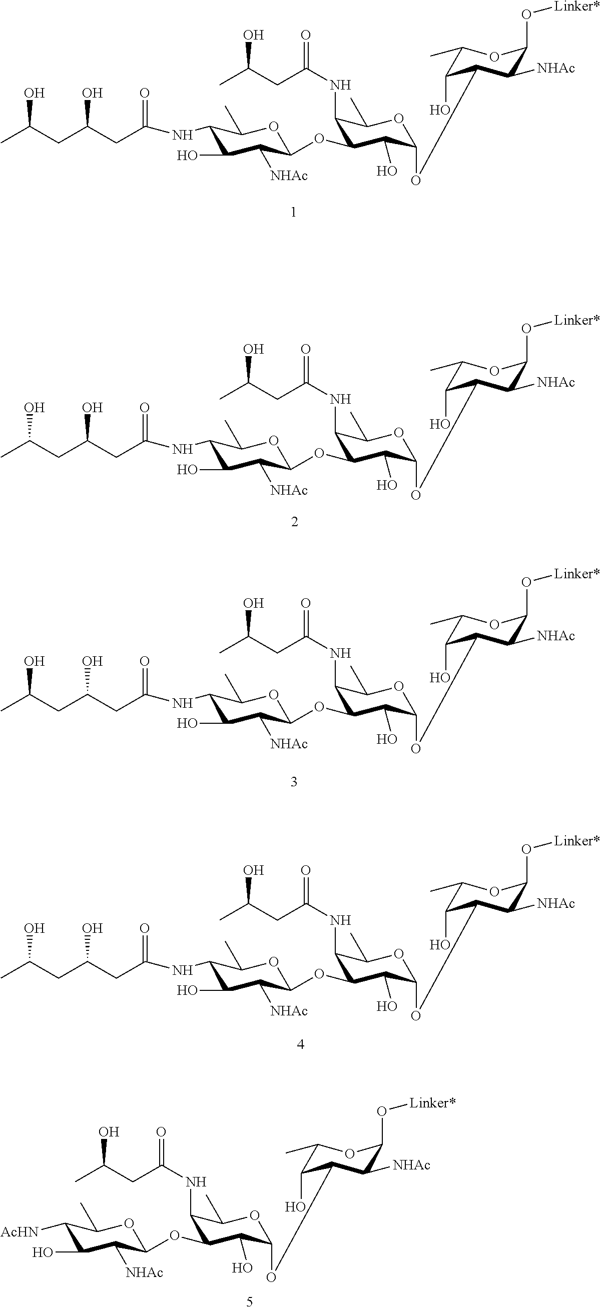
wherein Linker* is —(CH₂)ₙNH₂ or —(CH)ₙSH, wherein n=1-25;
the structures of the three monosaccharide building blocks are respectively represented by Formulae (6) to (8), and the structures of the five carboxylic acid derivatives are respectively represented by Formulae (9) to (13): 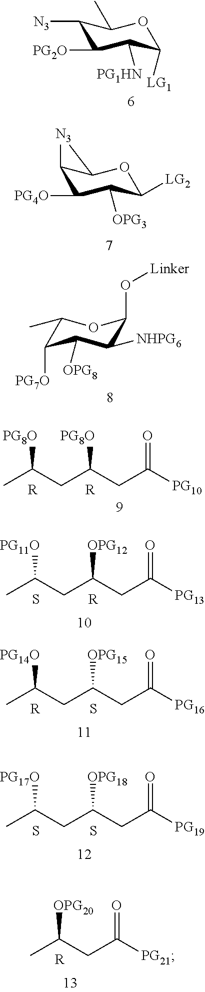
PG₂, PG₃, PG₄, PG₆, and PG₇ are temporary hydroxyl protecting groups each independently selected from benzyl, 2-naphthylmethyl, tert-butyldimethylsilyl, tert-butyldiphenylsilyl, and triethylsilyl;
PG₈, PG₉, PG₁₁, PG₁₂, PG₁₄, PG₁₅, PG₁₇, PG₁₈, and PG₂₀ are temporary hydroxyl protecting groups each independently selected from benzyl, 2-naphthylmethyl, acetyl, benzoyl, neopentyl, 9-pentamethoxycarbonyl, and 2-p-methoxybenzyl;
PG₁₀, PG₁₃, PG₁₆, PG₁₉, and PG₂₁ are each independently selected from one of hydroxyl, chlorine, bromine, fluorine, and C1-4 alkoxy;
PG₁ is a temporary amino protecting group selected from trichloroacetyl, dichloroacetyl, and chloracetyl;
PG₅ is a temporary amino protecting group selected from acetyl, trichloroacetyl, dichloroacetyl, chloracetyl, trichloroethoxycarbonyl, phthaloyl, 9-fluorenylmethoxycarbonyl, and tert-butyloxycarbonyl;
Linker is —(CH₂)ₙN—Y₁Y₂ or —(CH)ₙS—Y₁, wherein n=1-25, and Y₁ and Y₂ are one of hydrogen, acyl, benzyl, 2-naphthylmethyl, and benzylmethoxycarbonyl;
a leaving group LG₁ is N-phenyltrifluoroacetimidate;
a leaving group LG₂ is selected from one of trichloroacetimidate, N-phenyltrifluoroacetimidate, methylthio, phenylselenyl, ethylthio, phenylthio, p-tolylthio, and dibutylphosphono;
wherein the chemical synthesis method comprises the following steps:
(1) construction of disaccharide acceptor: removing a hydroxyl protecting group PG₆ at a site 3 of monosaccharide building block 8 to obtain acceptor 14; enabling the acceptor 14 and monosaccharide building block 7 to undergo a glycosylation reaction in a mixed system of anhydrous dichloromethane and diethyl ether to obtain disaccharide 15; reducing an azide group in the disaccharide 15 to an amino group by a reducing agent, and adding compound 13 for amidation to obtain compound 16; removing a hydroxyl protecting group PG₄ at a site 3 of the monosaccharide building block 7 in compound 16 to obtain disaccharide acceptor 17; 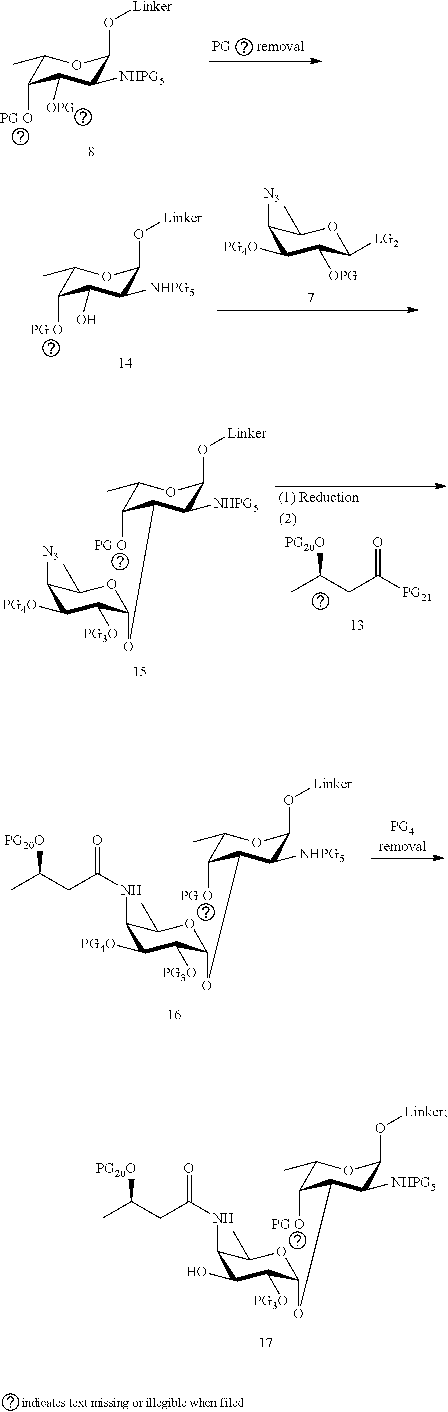
(2) construction of target trisaccharide:
enabling the disaccharide acceptor 17 and monosaccharide building block 6 to undergo a glycosylation reaction under the action of an activating agent, and controlling the temperature of the reaction to gradually rise from 0° C. to room temperature to react to obtain trisaccharide 18; reducing an azide group of the trisaccharide 18 by a reducing agent, and adding any one of the carboxylic acid derivatives represented by Formulae (9) to (12) for amidation to correspondingly obtain compounds 19 to 22; enabling the compounds 19 to 22 to undergo catalytic hydrogenation for deprotection to obtain target compounds 1 to 4;
or, enabling the trisaccharide 18 to undergo reductive acylation to convert an azide group to an acetylamino group, followed by catalytic hydrogenation for deprotection to obtain target compound 5; 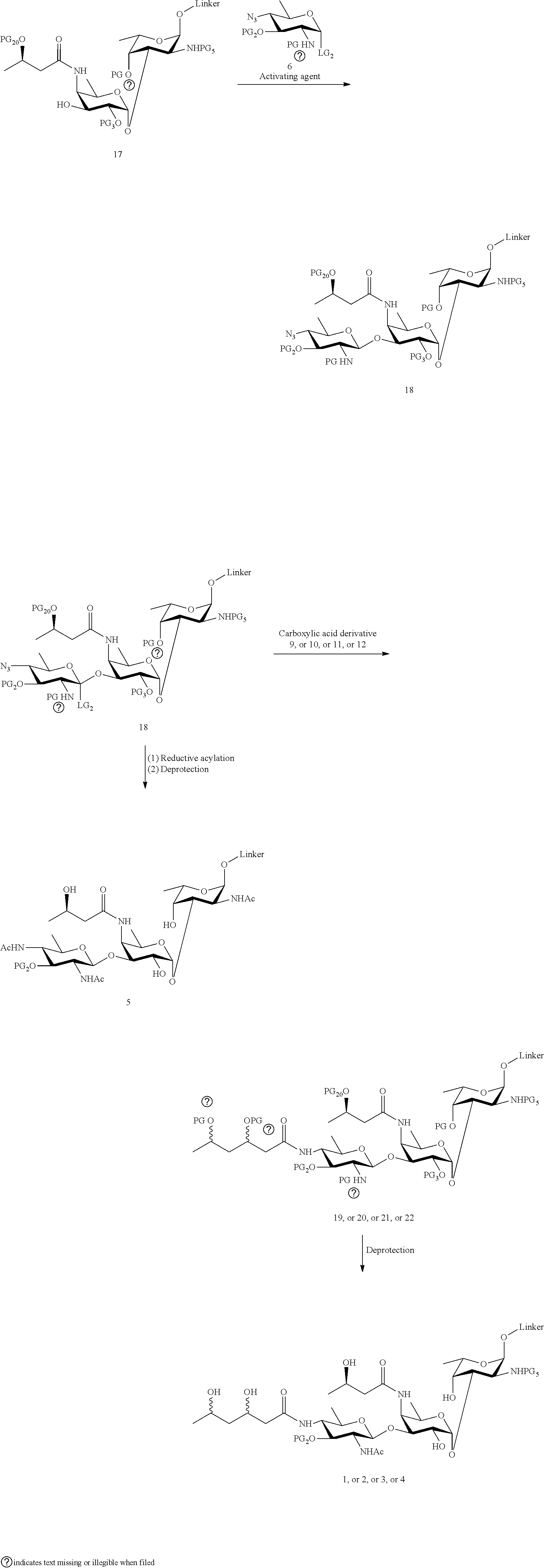
PGₐ and PG_{b} are temporary hydroxyl protecting groups each independently selected from benzyl, 2-naphthylmethyl, acetyl, benzoyl, neopentyl, 9-pentamethoxycarbonyl, and 2-p-methoxybenzyl.

2. The chemical synthesis method according to claim 1, wherein in step (1), the concentration of the glycosylation reaction is 0.01 mol/L-0.1 mol/L; an activating agent is used for the glycosylation reaction, and the activating agent is one of TMSOTf, NIS/TMSOTf, and NIS/TfOH; and the molar ratio of the monosaccharide building block 7 to the acceptor 14 is (1-3):1 or 1:(1-3).

3. The chemical synthesis method according to claim 1, wherein in step (1), specific conditions for the glycosylation reaction are: the monosaccharide building block 7 and the acceptor 14 are dissolved in a mixed solvent of dichloromethane and diethyl ether and stirred under the protection of argon, a molecular sieve is added, the reaction temperature is −20° C. to 0° C., 0.1-0.3 equivalents of the activating agent relative to the molar weight of the monosaccharide building block 7 is added, and the reaction time is 2 hours-8 hours.

4. The chemical synthesis method according to claim 1, wherein in step (1), the reducing agent for reducing the azide group in the disaccharide 15 is one of zinc powder, triphenylphosphine, 1,3-propanedithiol, lithium aluminum hydride, trimethylphosphine, stannous chloride dihydrate, sodium borohydride, and sodium cyanoborohydride.

5. The chemical synthesis method according to claim 1, wherein in step (2), the concentration of the glycosylation reaction is 0.01 mol/L-0.1 mol/L; and the activating agent is one of TMSOTf, NIS/TMSOTf, and NIS/TfOH.

6. The chemical synthesis method according to claim 1, wherein in step (2), the glycosylation reaction is performed in a solvent, and the solvent comprises one or more of anhydrous dichloromethane, diethyl ether, toluene, methanol, tetrahydrofuran, acetonitrile, N,N-dimethylformamide, or water; and the molar ratio of the monosaccharide building block 6 to the disaccharide acceptor 17 is (1-3):1 or 1:(1-3).

7. The chemical synthesis method according to claim 1, wherein in step (2), conditions for the glycosylation reaction comprise: the disaccharide acceptor 17 and the monosaccharide building block 6 are dissolved in a dichloromethane solvent, a molecular sieve is added, 0.2-1 equivalent of the activating agent relative to the molar weight of the disaccharide acceptor 17 is added, the temperature of the reaction is controlled to gradually rise from 0° C. to room temperature, and the reaction time is 2 hours to 8 hours.

8. The chemical synthesis method according to claim 1, wherein in step (2), the reducing agent is 1,3-propanedithiol; and the process of reduction further comprises addition of a condensing agent, and the condensing agent is selected from HATU and EDC.

9. The chemical synthesis method according to claim 1, wherein in step (2), a catalyst used for the catalytic hydrogenation is a 10% palladium-on-carbon catalyst or palladium hydroxide.

10. An application of the method according to claim 1 in preparation of a glycan microarray or V. cholerae glycoconjugate, the application comprising the following processes:
S1: preparing oligosaccharide fragments of V. cholerae serotype O100 O-antigen with Linker*; and
S2: then binding the linkers of the obtained oligosaccharide fragments to a microarray or carrier protein to obtain a corresponding glycan microarray or V. cholerae glycoconjugate.
